(19) United States
(12) Patent Application Publication
(10) Pub. No.: US 2003/0170756 A1
(43) Pub. Date: Sep. 11, 2003

(54) Treatment of tumor cells for use in immunotherapy of cancer

(75) Inventor: David Berd, Wyncote, PA (US)

Correspondence Address: DARBY & DARBY P.C., P. O. BOX 5257, NEW YORK, NY, 10150-5257, US

(73) Assignee: Thomas Jefferson University, Philadelphia, PA

(21) Appl. No.: 10/357,110

(22) Filed: Feb. 3, 2003

Related U.S. Application Data
(60) Provisional application No. 60/354,094, filed on Feb. 1, 2002.

(51) Int. Cl. A01N 1/02; G01N 33/574

(52) U.S. Cl. 435/7.23; 435/2

(57) ABSTRACT

A method comprising exposing tumor cells to ethanol has been found to preserve the tumor cells during storage. As compared to control cells, tumor cells are preserved for a longer time, and retain display of antigen. In a specific embodiment, modified or unmodified cells are exposed to a concentration of about 37.5% (v/v) ethanol for a period of about 10 minutes at about 40° C. Methods of storing haptenized tumor cells and vaccine preparations are also provided. It has also been found that tumor cell vaccines which comprise mainly dead or non-Trypan Blue-excluding cells can have retained or even improved antigenicity as compared to live cells. Methods of preparing and using such vaccines are also described.

This application claims priority from U.S. Provisional Application Serial No. 60/354,094, filed Feb. 1, 2002, which is hereby incorporated by reference in its entirety.

### FIELD OF THE INVENTION

The invention relates to compositions comprising a tumor cell treated for preservation, sterility, or both. The tumor cell compositions are particularly suitable for immunotherapeutic vaccine. Haptenized tumor cell preparations are especially advantageous.

### BACKGROUND OF THE INVENTION

In blood transfusion, bone marrow transplantation, immunotherapeutic vaccine preparation, or other cell preparations ex vivo, one of the principal problems encountered is that of the preservation of cells. It is critical to be able to preserve cells, under good conditions of viability, for time periods compatible with clinical production and storage, and to make it possible to analyze cell preparations. The most commonly used method of long-term preservation of cells is to freeze and subsequently thaw them. However, during the freezing of cells, lysis of cells and loss of cell integrity may occur. This problem can be even more complex when the cells have been modified or altered prior to preservation, and when the cells are obtained by proteolytic digestion of a tissue or tumor specimen.

Preservation of cells under less extreme conditions, for example on ice (about 0° C.), refrigerated (about 4° C.), or at room temperature, prior to use, is also difficult.

### Immunotherapy

The preservation of cells, especially their antigenicity, is important is in immunotherapy of cancer using tumor cells. The aim of the immunotherapy is to evoke an immune response to the tumor, or to vaccinate against new tumors, by administering tumor cells to the cancer patient. The tumor cells in the composition should contain antigens that are also present in the tumor to be treated, so that the immune response elicited against the antigens in the composition is effected against the tumor. Generally, the cells are recovered from tumors, suspended in a cryopreservation medium and frozen until used for the vaccine preparation. When needed, the cells are thawed, and then stored at temperatures ranging from about 0° C. (on ice) to room temperature until administration.

Immunotherapy regimens using unmodified intact tumor cells prepared from tumors taken from the patient, i.e., autologous tumor cells, have been extensively described in the literature (see, e.g., Berd et al., Cancer Research 1986;46:2572-2577; Hoover et al., Cancer 1985;55: 1236-1243; and U.S. Pat. No. 5,484,596 to Hanna et al.). Alternative vaccine compositions based on disrupted cells have also been suggested including, e.g., tumor membranes (see, e.g., Levin et al., In: Human Tumors in Short Term Culture: Techniques and Clinical Applications, P. P. Dendy, Ed., 1976, Academic Press, London, pp. 277-280) or tumor peptides extracted from tumors (see, e.g., U.S. Pat. No. 5,550,214 to Eberlein, and U.S. Pat. No. 5,487,556 to Elliot et al.). The tumor cells can also be modified in some manner to alter or increase the immune response (see, e.g., Hostetler et al., Cancer Research 1989;49:1207-1213, and Muller et al., Anticancer Research 1991;1 1:925-930).

### Haptenized Tumor Cell Vaccines

One particular form of tumor cell modification that has a pronounced effect on immunotherapy is coupling of a hapten to the tumor cells. An autologous whole-cell vaccine modified with the hapten dinitrophenyl (DNP) has been shown to produce inflammatory responses in metastatic sites of melanoma patients. Adjuvant therapy with DNP-modified vaccine produces markedly higher post-surgical survival rates than those reported after surgery alone. U.S. Pat. No. 5,290,551 to Berd discloses and claims vaccine compositions comprising haptenized melanoma cells. Melanoma patients who were treated with these cells developed a strong immune response. This response can be detected in a delayed-type hypersensitivity (DTH) response to haptenized and non-haptenized tumor cells. More importantly, the immune response resulted in increased survival rates of melanoma patients.

Haptenized tumor cell vaccines have also been described for other types of cancers, including lung cancer, breast cancer, colon cancer, pancreatic cancer, ovarian cancer, and leukemia (see International Patent Publication Nos. WO 96/40173 and WO 00/09140, and U.S. Pat. No. 6,333,028, and the associated techniques and treatment regimens optimized (see International Patent Publication Nos. WO 00/38710, WO 00/31542, WO 99/56773, WO 99/52546, and WO 98/14206). For example, it has been shown that the addition of human serum albumin (HSA) increases the stability of haptenized tumor cell preparations (see WO 00/29554 and U.S. Pat. No. 6,248,585).

It has also been found that haptenization of tumor cell extracts such as plasma membranes and peptides can yield potent immunotherapy vaccines (see International Patent Publication Nos. WO 96/40173 and WO 99/40925, both by Berd et al.).

For haptenized vaccines, the search for storage conditions that preserve the stability of the haptenized cells or extracts also have to take into account that some haptenization reactions may alter or affect the cell viability or integrity. Previous work has suggested that if no measures are taken to increase the stability of haptenized melanoma vaccine preparations, they might have a cell integrity duration of less than four hours after hapten modification. Also, some haptens or hapenization procedures render the cells more fragile than others. For example, while preparations of DNP-modified cells can be stable for at least 18 hours when stored at 4° C., some procedures for sulfanilic acid (SA) conjugation render the cells more fragile, and the SA-modified cells may in some cases only be stable for less than 2 hours at 4° C.

However, whether utilizing modified or unmodified tumor cells, in order to elicit a successful immune response against the tumors of the patient after administration, the amount and antigenicity of the antigens in the tumor cell composition should be retained during preparation and storage of the composition. The tumor antigens should also remain associated with the cells.

Thus, there is a need in the art for an effective treatment for cells to be stored and preserved prior to delivery as an immunotherapy vaccine. There is also a need for a treatment that preserves the antigenicity of such vaccines prior to administration, and methods for designing tumor cell preparations and formulations to obtain optimal immune response. The present invention advantageously addresses these and other needs in the art.

### SUMMARY OF THE INVENTION

The present invention advantageously provides a method of treating tumor cells for their preservation and/or storage prior to use in anti-tumor vaccines. Thus, in a first embodiment, the invention provides a method of treating a tumor cell comprising exposing the tumor cell to a preserving agent such as, for example, ethanol, isopropanol, or paraformaldehyde, for a period of time and at a concentration effective to stabilize the tumor cell until administration to the patient. The tumor cell may be modified or unmodified. One type of modified cells that are suitable for use in the present invention are haptenized cells, or cells intended for haptenization.

The invention also provides a method of preserving tumor cells, which method comprises contacting the tumor cells with ethanol at a concentration effective to preserve the tumor cells, whereby the tumor cells are better preserved than the same type of tumor cells incubated in control medium without ethanol for the same period of time and at the same temperature. The concentration of ethanol can be within the range of about 22.5% to about 75% by volume, more preferably about 37.5% by volume. The method may comprise contacting the tumor cells with ethanol for a period of about 2 minutes to about 24 hours at a temperature within the range of about 0° C. to about 20° C., more preferably for a period of about 10 minutes at a temperature of about 4° C. In a preferred embodiment, the tumor cell preservation comprises preservation of antigenicity. Alternatively, the tumor cell preservation comprises preservation of the number of cells. The method of the invention can be used on tumor cells selected from, for example, melanoma cells, ovarian cancer cells, colorectal cancer cells, small cell lung cancer cells, kidney cancer cells, breast cancer cells, and leukemia cells. More preferably, the cells are melanoma cells or ovarian cancer cells. In a particular embodiment, the tumor cells are conjugated to a hapten. The hapten may be selected from DNP, TNP, and sulfanilic acid, or combinations thereof.

In addition, the invention provides a composition comprising tumor cells for use in a vaccine and a concentration of ethanol effective to preserve the tumor cells. Preferably, the concentration of ethanol is within the range of about 22.5% to about 75% by volume, more preferably about 37.5% by volume. The temperature of the composition can be within the range of about 0° C. to about 20° C., more preferably about 4° C. In preferred embodiments, the concentration of ethanol is effective to preserve the antigenicity of the tumor cells and/or the number of tumor cells. The tumor cells may be, for example, melanoma cells, ovarian cancer cells, colorectal cancer cells, small cell lung cancer cells, kidney cancer cells, breast cancer cells, or leukemia cells. Preferably, the tumor cells are melanoma cells or ovarian cancer cells. In a particular embodiment, the tumor cells are conjugated to a hapten. The hapten may, for example, be selected from DNP, TNP, and sulfanilic acid, or combinations thereof.

The invention also provides for a tumor cell vaccine comprising (i) dead autologous tumor cells; and (ii) an adjuvant, wherein the vaccine is essentially free of live autologous tumor cells of the same tumor type. Preferably, the antigenicity of the autologous tumor cells is no less than the antigenicity of live autologous tumor cells of the same tumor type. The tumor cells can be, for example, melanoma cells, ovarian cancer cells, colorectal cancer cells, small cell lung cancer cells, kidney cancer cells, breast cancer cells, and leukemia cells. Preferably, the tumor cells are melanoma or ovarian cancer cells. In one embodiment, the tumor cells are conjugated to a hapten. The hapten can be, for example, DNP, TNP, or sulfanilic acid, or a mixture thereof.

The invention also provides for a method for treating cancer in a subject, comprising administering a vaccine comprising an adjuvant and autologous tumor cells which have been treated to render them dead, wherein the vaccine is essentially free of live autologous tumor cells of the same tumor type. In one embodiment, the tumor cells have been treated with ethanol, preferably ethanol within the range of about 22.5% to about 75% by volume, more preferably about 37.5% by volume. The tumor cells can be conjugated to at least one hapten. The hapten can be at least one hapten selected from the group consisting of DNP, TNP, and sulfanilic acid. For example, the tumor cells can comprise a first fraction of tumor cells conjugated to DNP, and a second fraction of tumor cells conjugated to sulfanilic acid.

The present invention will be further explained by the Drawings, Detailed Description, and Examples.

### BRIEF DESCRIPTION OF THE DRAWINGS

FIG. 1. This figure shows flow cytometry evaluation using an anti-HLA class I antibody of ethanol-treated, bi-haptenized, melanoma cells. Three parts of a 0% (A; control), 50% (B), or 70% (C) ethanol solution was added to one part mixed-haptenized tumor cell suspension (see Example 2).

FIG. 2. This figure shows flow cytometric analysis of unmodified cells (A) and ethanol-treated and sulfanilic acid (SA)-modified melanoma cells (B). Forward light scatter, an indication of cell diameter, was measured.

FIG. 3. This figure shows a flow-cytometric comparison between unmodified and fixed (A), unmodified unfixed (B), DNP-modified and fixed (C), and SA-modified and fixed melanoma cells (D). An antibody against HLA class I antigen was used in the analysis.

FIG. 4. This figure displays flow cytometry histograms showing the effect of various concentrations of ethanol on cells. An antibody against HLA class I antigen was used in this analysis.

FIG. 5. This figure shows the number of preserved cells in various ethanol-preserved preparations of mixed-haptenized melanoma cells, after certain periods of incubation at 4° C.

FIG. 6. This figure shows the number of preserved cells in three preparations of mixed-hapterized melanoma cells after up to 7 days of incubation at 40° C.

FIG. 7. This figure shows the antigen-preservation of mixed-haptenized ethanol-fixed melanoma vaccine, by flow-cytometric analysis using antibodies directed against the haptens DNP and SA (A and B, respectively), the melanoma-associated antigens S100 and GD3 (C and D, respectively), and HLA class I antigen (E). (F) is a control. Ethanol-treated cells were frozen for up to two months, and then thawed for analysis.

FIG. 8. This figure shows inhibition of proliferation of mixed-haptenized and ethanol-fixed melanoma cells. The proliferation of various preparations of unmodified cells were compared to cells that had been fixed, and to cells that had been both mixed-haptenized and fixed.

FIG. 9. This figure shows the delayed-type hypersensitivity response (DTH) measured in patients immunized with DNP-modified melanoma cells to DNP-modified tumor cells (A) and unmodified tumor cells (B). The DTH response to ethanol-fixed cells was compared to that of untreated or “fresh” cells for both types of cells.

### DETAILED DESCRIPTION

As described herein, the present invention contemplates tumor cell preparations and vaccines in which the tumor cells are dead and, e.g., permeable to Trypan Blue or other supravital agents, and have a substantially retained or improved antigenicity as compared to a vaccine comprising live and/or Trypan Blue-excluding cells. Such vaccines may or may not be haptenized. The preparation of such tumor cell vaccines include a treatment step wherein the treatment leads to permeabilized or dead cells while at least retaining antigen expression or display on the tumor cell surface. Advantageously, the treatment also has an additional benefit, such as leading to improved sterility, purity, or preservation of the vaccines. Exemplary but non-limiting treatments include very high doses of radiation (e.g., 100,000 cGy) which can be bactericidal; heating (e.g., to ≧60° C. or greater) to kill certain bacteria or viruses; treatment with alcohols such as ethanol or isopropanol that can be bactericidal while maintaining antigen display; treatment with other chemicals than alcohols, e.g., paraformaldehyde, which is known to maintain antigen display; and purification on polymyxin columns to remove endotoxins. While it is often desirable to remove treatment agents such as alcohols from the tumor cell vaccine after the treatment step, the treatment agent can also be a pharmaceutically acceptable agent which can remain in the vaccine. Examples of such agents are preservatives such as, e.g., sodium azide or merthiolate. The experimental parameters of the treatment step, including concentration of agent, length of exposure to the tumor cells, and optional purification, can be determined by routine experimentation. For example, the optimization and evaluation techniques used for ethanol treatment, described in detail herein, can be used for other agents as well.

Thus, the present invention advantageously provides new preservation methods which stabilizes tumor cells, including modified tumor cells such as haptenized cells, for storage. The preserved cells are preferably stored at between about 0° C. (on ice) and 20° C. (at room temperature) prior to delivery to the patient. In one embodiment, the method for the preservation and/or storage of tumor cells comprises contacting the cells with an optimized concentration of ethanol. After ethanol treatment, most or all of the preserved cells are dead, and the tumor cell composition essentially free of live cells. The preservation method of the invention is suitable for treatment of any tumor cell, such as, e.g., haptenized or non-haptenized tumor cells derived from melanoma, ovarian cancer, small cell lung cancer, colon cancer, leukemia, or lymphoma.

After the preservation treatment step, the cells may be used for preparing a tumor cell vaccine for administration to a patient in need thereof. The preservation method of the invention is particularly advantageous for such applications, since preserved cell can be maintained a longer time in solution without losing antigenicity or vaccine potency, thus permitting a longer period of time for quality assurance (QA) and quality control (QC) of the vaccine before administration to the patient.

Yet another advantage of the method of the invention using, e.g., ethanol treatment, is its bactericidal effect. Bacterial contamination can be a problem when preparing vaccines or other medications from tissues. The anti-bacterial effect of treatment with ethanol, isopropanol, irradiation, heat, etc., treatment can therefore improve sterility of tumor cell vaccines, or even obviate the necessity for additional treatment steps to sterilize tumor cell preparations.

Cells treated with the optimized concentration of preserving agent remain substantially intact and preserve antigen display on the tumor cell surface, as determined by flow cytometry, to a greater extent than that of control cells that have not been treated with the agent. For example, greater than 10% of ethanol-treated tumor cells are preserved during storage for a three-day period at about 4° C., as compared to the initial number of cells after ethanol treatment. Preferably, greater than about 25% of the cells are preserved; more preferably, more than 50% of the cells are preserved, and, even more preferably, 75% of the cells are preserved. For SA-modified tumor cells not treated with ethanol, typically 90% of the cells can be lost, i.e., lysed, after 2-4 hours incubation at 4° C. Preferably, the preservation of tumor cells treated with ethanol is greater than the preservation of the same kind, number, and concentration of tumor cells incubated in control medium without ethanol for the same period of time and at the same temperature.

The treatment step may result in loss of cells, but the remaining cells are substantially intact and retain their display or accessibility of relevant cell surface antigens. Moreover, they are stable for at least 3 days at 4° C., and the shelf-life of the treated cells can be extended by freezing. This preparation has the following advantages over prior art preparations of modified or unmodified tumor cells: (1) treatment prolongs the shelf life; (2) irradiation is not necessary; and (3) cell counting is made easier because differentiation between “dead” and “live” tumor cells is moot. The opportunity to exclude irradiation of tumor cell vaccines is a particularly attractive feature of the invention, since irradiation has been a technically cumbersome and economically burdensome necessity in previous procedures to render the cells non-proliferative. Essentially all treated cells of the invention take up Trypan Blue or other supravital dyes to some extent but have substantially intact membranes, preserved shape, and retain surface antigens.

Furthermore, according to the present invention, autologous tumor cell vaccines comprising dead or non-Trypan Blue excluding cells, or consisting wholly of dead cells or Trypan Blue excluding cells are equally effective, in some cases even better, in eliciting an immune response against a tumor as tumor cell vaccines comprising live cells. See, e.g., Examples 6, 9, and 10. Thus, according to one embodiment, the invention provides tumor cell vaccines wherein substantially all cells are dead or permeable to Trypan Blue, and essentially free of live, Trypan Blue-excluding cells, as well as methods of preparing such vaccines and treating cancer patients with such vaccines.

The various aspects of the invention will be set forth in greater detail in the following sections, directed to suitable media and formulations for preserving haptenized tumor cells. This organization into various sections is intended to facilitate understanding of the invention, and is in no way intended to be limiting thereof.

### Definitions

The following defined terms are used throughout the present specification, and should be helpful in understanding the scope and practice of the present invention.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

If not otherwise stated, the concentration of a liquid in a liquid mixture is given as percentage of the liquid in the total volume (% v/v) of the mixture, i.e., “by volume”. For example, a 3:1 mixture between 50% ethanol and HBSS would lead to a 37.5% v/v ethanol solution, or 37.5% ethanol by volume.

A “formulation” refers to an aqueous medium or solution for the preservation of haptenized tumor cells, which is preferably directly injectable into an organism. An aqueous buffer will include salts or sugars, or both, at about an isotonic concentration. The formulation may further comprise ethanol, as described herein. “Human serum albumin” or “HSA” refers to a non-glycosylated monomeric protein consisting of 585 amino acid residues, with a molecular weight of 66 kD. Its globular structure is maintained by 17 disulphide bridges, which create a sequential series of 9 double loops (Brown, “Albumin structure, function and uses”, Rosenoer, V. M. et al. (eds.), Pergamon Press:Oxford, pp. 27-51, 1977). HSA may also be called human plasma albumin.

A “live” cell means a cell that has an intact cell, plasma, or “outer” membrane as assessed by exclusion of a supravital dye such as Trypan Blue. A live cell may be capable of growth or maintenance, and division or multiplication, or attenuated, i.e., incapable of division and multiplication. A cell can be rendered attenuated by, for example, irradiation.

“Dead” cells means cells that do not exclude supravital dyes such as Trypan Blue, propidium bromide, or ethidium bromide, as assessed in an exclusion experiment (see, e.g., Methods In Analysis Of Apoptosis And Cell Necrosis by Darzynkiewicz Z., In: The Purdue Cytometry CD-ROM Vol 3, J. Parker, C. Stewart, Guest Eds.; J. Paul Robinson, Publisher, Purdue University, West Lafayette, 1997). Dead cells are incapable of division or multiplication. A “dead” cell can be prepared by, e.g., ethanol treatment of a live cell. A dead cell may appear intact, e.g., by microscopic inspection, meaning that the cellular shape resembles that of a live cell. A “fixed” cell is one example of a dead cell.

A “lysed” cell is no longer intact, meaning that the cellular shape does not resemble that of a live cell.

The “total” number of tumor cells in a preparation means the sum of live and dead tumor cells in the preparation.

A “preserved” cell is a cell which is not lysed. A preserved cell can be live or dead. The cell may or may not exclude Trypan Blue, but retains its antigenicity over time better than a cell which is not similarly preserved. “Preservation” of cells can be expressed as the percentage of cells remaining after a certain period of time following ethanol treatment of the cells according to the method of the invention. Thus, about 90% of the cells being preserved over a period of 1 day (i.e., 24 hours) means that the number of “non-lysed” cells in the preparation after 1 day storage is about 90% of the number of “non-lysed” cells in the preparation just after ethanol treatment.

Treatment with ethanol can lead to “fixed” cells. Ethanol-treatment can therefore also be termed “fixation”.

“Antigenicity” means the ability of a tumor cell to evoke an immune response directed to the tumor cell. Generally, antigenicity is higher for a tumor cell that comprises tumor-specific antigens than a tumor cell which does not comprise, or comprises a lower amount of, tumor-specific antigens. Antigenicity can be measured by, for instance, DTH-testing, or by measuring the number of tumor cell-associated antigens using, e.g., FACS analysis with antibodies directed against the tumor-associated antigens.

The term “cell recovery” or “cell recovery rate” is a measure of how many cells are substantially intact, has a shape corresponding to or resembling that of a live cell, and/or has preserved antigenicity, after a certain period of storage or incubation. When calculating cell recovery, the number of cells at a certain time point or after a certain preparation step is related to the number of cells at a reference time point or prior to the preparation step in question.

The phrase “pharmaceutically acceptable” refers to molecular entities, at particular concentrations, and compositions, that are physiologically tolerable and do not typically produce an allergic or similar untoward reaction, such as gastric upset, fever, dizziness and the like, when administered to a human or non-human animal. Preferably, as used herein, the term “pharmaceutically acceptable” means approved by a regulatory agency of the Federal or a state government, or listed in the U.S. Pharmacopoeia or other generally recognized pharmacopoeia for use in humans or non-human animals.

A “subject” is a human or a non-human animal who may receive haptenized tumor cells formulated in a composition of the invention. Non-human animals include domesticated pets, such as cats and dogs; farm animals, such as horses, cows, pigs, sheep, and goats; laboratory animals, such as mice, rats, guinea pigs, and rabbits; etc.

An “anti-tumor response” is at least one of the following: tumor necrosis, tumor regression, tumor inflammation, tumor infiltration by activated T lymphocytes, activation of tumor infiltrating lymphocytes, delayed-type hypersensitivity (DTH) response, or a clinical response. Clinical response criteria for anti-tumor response resulting from treatment according to the present invention include complete, partial, or mixed response, as well as stable disease. Other clinical responses that may be observed following treatment according to the invention is prolongation of time to relapse, or prolongation of survival.

A “formulation” refers to an aqueous medium or solution for the preservation or administration, or both, of haptenized tumor cells, which is preferably directly injectable into an organism. The aqueous medium can include salts or sugars, or both, at about an isotonic concentration.

A “vaccine composition” is a composition as set forth previously further comprising an adjuvant, including an immunostimulatory cytokine or lymphokine.

The terms “vaccine”, “immune therapy” and “immunotherapy” are used herein interchangeably to administration of a composition comprising a tumor cell preparation (preferably haptenized) to treat a cancer, e.g., after surgical resection of the tumor.

“Efficacy of an immunotherapy” is the degree to which the immunotherapy elicits am anti-tumor response in an individual subject, or the percentage of subjects in which an anti-tumor response develops as a result of treatment. Preferably efficacy is determined by composition to controls that harbor the spontaneous tumor but receive either no therapy, sham therapy, or an alternative therapy.

A “tumor cell preparation” refers to isolated or purified tumor cells for inclusion in a composition. “Hapten modified” means that the tumor cells are chemically coupled (conjugated) to a hapten, as that term is understood immunology.

As used herein, a “bi-haptenized”, “multi-haptenized”, or “mixed haptenized” tumor cell preparation means a composition comprising two or more tumor cell preparations, in which each tumor cell preparation is differently haptenized.

The term “differentially haptenized” as used herein refers to mixture of at least two haptenized tumor cells, wherein a first cell was haptenized under a particular condition or using a particular reagent and a second cell was haptenized under a different condition or using a different reagent. The conditions or reagents may differ so that, for example, different amino acids are haptenized on the proteins of the first and second tumor cells, and/or that the hapten attached to the first cell is different from the hapten attached to the second cell.

The term “treat” means to attempt to elicit an anti-tumor response against cells of the tumor, i.e., the cancer. An anti-tumor response includes, but is not limited to, increased time of survival, inhibition of tumor metastasis, inhibition of tumor growth, tumor regression, and development of a delayed-type hypersensitivity (DTH) response to unmodified tumor cells.

As used herein, the term “control” generally describes a cell or cells not treated with ethanol. More preferably, a control describes a composition which in essentially all other aspects than ethanol treatment has been exposed to the same conditions, and is stored in the same buffered medium and additional components.

### Treatment with Ethanol or Other Agents

As noted above, and demonstrated in the Examples, infra, it has been unexpectedly discovered that exposure of tumor cells to an appropriate concentration of a preserving agent such as ethanol in a buffered cultured medium, preferably HBSS, greatly increases cell preservation and antigenicity. This is especially advantageous for tumor cells for use in immunotherapy vaccine preparations. Accordingly, depending on the specific tumor cells to be stored, and their modification, if any, one of ordinary skill in the art can test for the optimum concentration of ethanol or other preserving agent for, as well as the duration of, such a treatment step, as exemplified infra. Such a concentration can be one that yields an increase in cell preservation relative to a control for stored tumor cells. In addition, such a concentration can be one that retains the amount of antigen-displaying cells relative to a control. Preferably, the increase in preservation of the number of cells is statistically significant. In a specific embodiment, the yield of intact cells after treatment is at least about 10%, more preferably at least about 20%, and even more preferably, at least about 50%. In a preferred embodiment, the cells are then stored in 1% HSA in HBSS.

After treatment, the cells are stable or preserved in that at least about 30%, preferably at least about 50%, and even more preferably at least about 80%, of the treated cells can be present after about 3 days of storage at 4° C., and have substantially retained antigen content. See also Table 2 in the Examples. By contrast, in one example, about 90% of SA-modified cells not exposed to the preserving agent ethanol were lost (i.e., lysed) during 4 hours of storage at 4° C. In experiments using SA-modified cells, the recovery of total cells (including dead cells) is rarely more than 30% after 4 hours storage at 4° C. Thus, preservation of antigen-displaying or antigen-associated cells can be substantially improved by treatment with an agent such as ethanol. Preferably, the preservation of a tumor cell subjected to treatment with an agent is greater than the same kind of tumor cells incubated in control medium without the agent for the same period of time, at the same temperature.

The following is a description of one treatment according to the invention, using ethanol as preserving agent. Tumor cells suspended in a suitable medium, such as, but not limited to, HBSS, and are kept on ice, at about 0° C. to 10° C., or at about 4° C. Optionally, the medium contains HSA at a concentration of, for example, 1% (weight to volume). Next, ethanol is added to the cells at a suitable final concentration (see below). In one embodiment, 3 ml of ice-cold ethanol solution (50% v/v) are added per each ml of tumor cell suspension. The ethanol can be added to each tube while vortexing at low speed. The tubes are thereafter incubated in the presence of ethanol. Suitable incubation time and temperature can be determined experimentally for different tumor cell preparations. For example, it has been found that a 10 minute incubation at 4° C. is suitable for mixed-haptenized cells (see Examples 1-3). The cells are thereafter pelleted by centrifugation, e.g., by spinning at 1100 RPM for 7 minutes. The supernatant is aspirated to remove the ethanol-containing supernatant, and the cells washed in medium. For example, 5×10⁶ cells can be resuspended in 10 ml HBSS+1% HSA, and pelleted again by spinning at 1100 RPM for 7 minutes. This washing procedure can be repeated if necessary. After washing, the cells are pelleted, the supernatant aspirated, and the cells resuspended in the desired medium. For example, 5×10⁶ cells can be resuspended in 2 ml Hanks+1% HSA (se also “Formulations”, below). Preferably, the cells are stored in a medium suitable for administration to a subject. In another embodiment, the cells are stored in a medium suitable for cryopreservation, and cryopreserved (see below) until needed.

Any ethanol concentration effective to preserve the tumor cells may be used in this procedure, for example by varying either the ethanol concentration in the stock solution added to the HBSS solution, and/or by varying the amount of ethanol added to the HBSS solution. Generally, treatment with a solution containing more than 75% ethanol leads to fixation of cells, but also to loss of antigens. Thus, according to the invention, the cells are preferably incubated in about 5% to about 75% (v/v) ethanol. More preferably, the cell are incubated in about 20% to about 60% (v/v) ethanol, or, even more preferably, in about 25% to about 40% (v/v) ethanol. In a particularly preferred embodiment, the cells are incubated in about 30-40% (v/v) ethanol. In one specific embodiment, the cells are treated in no greater than about 52.5% (v/v) ethanol. In another specific embodiment, the cells are incubated in about 37.5% (v/v) ethanol. A suitable ethanol concentration is one that can fix the cells, maintain display or association of antigens, and prevent cell proliferation. In one embodiment, a suitable ethanol concentration has, in addition, a bactericidal effect.

The duration as well as the temperature of the ethanol treatment step may also have an impact on the preservation of the cells. Preferably, the ethanol exposure is conducted at room temperature or less, preferably at 10° C. or less, and even more preferably at about 4° C. or on ice. A period of incubation for about 10 minutes is suitable for mixed-haptenized cells. The optimal time period for modified or unmodified cells can be determined on a case-by-case basis using standard parameter-optimization procedures. The most suitable time of incubation would depend both on the modification and the type of tumor cell, as well as the temperature and ethanol concentration. Preferably, the cells are incubated for at least 10 seconds, preferably more than one minute, and, even more preferably, more than 2 minutes. In a preferred embodiment, the cells are incubated in ethanol for no more than 24 hours, preferably less than 1 hour, and even more preferably for about 10 minutes.

After the ethanol or other treatment step, the ethanol or other treatment agent is preferably, although not necessarily, substantially removed from the cells. This may be accomplished by, e.g., centrifugation, removal of the supernatant, and resuspending the cells in a suitable storage buffer as described above. As an alternative to centrifugation, the ethanol or other agent can be removed by dialysis, extraction, microfiber extraction, filtration, chromatography, evaporation, or other techniques known by those skilled in the art. Thereafter, the cells can be stored frozen, i.e., at less than 0° C., or not frozen, i.e., at above 0° C. A tumor cell composition which is stored frozen can be stored, e.g., at −10° C. to about −30° C., or, alternatively, in liquid nitrogen, which has a temperature of about −196° C. In one embodiment, the cells are first stored in a −70° C. or −86° C. freezer and then transferred to liquid nitrogen. A tumor cell composition which is stored at 0° C. or higher temperatures can be stored in a fridge, e.g., at between 0° C. to about 10° C. such as at about 4° C., or at room temperature, which corresponds to from about 15 to about 25° C.

The concentration of cells to be used during the ethanol or other treatment step can be determined experimentally depending on the type of cells or cell preparation used. However, a generally suitable concentration is between 10⁵-10⁸ cells, more preferably between 10⁶ to 10⁷ cells, and most preferably about 5×10⁶ cells, per milliliter solution. The solution is advantageously, although not necessarily, isotonic.

After ethanol or other treatment, at least the vast majority of the cells, preferably substantially all of the cells, take up Trypan Blue. However, by microscopic inspection, the cells are intact anatomically and/or has a shape resembling that of an intact cell. Generally, the treated cells are not easily distinguishable from living cells in the absence of Trypan Blue. The treated cells also retain antigen display to a substantial degree, as shown in the Examples.

For vaccines comprising haptenized tumor cells, the ethanol or other treatment is preferably, although not necessarily, conducted after haptenization.

### Tumor Cells

The tumor cells used in the present invention are prepared from tumor cells, e.g., obtained from tumors, or tissue or body fluids containing tumor cells, surgically resected or retrieved in the course of a treatment for a cancer. The ethanol-treated tumor cells are useful in the preparation of, e.g., tumor cell vaccines for treating cancer, including metastatic and primary cancers. If used in a tumor cell vaccine, the preserved tumor cells should be incapable of growing and dividing after administration into the subject, such that they are dead or substantially in a state of no growth. It is to be understood that “dead cells” means a cell which do not have an intact cell or plasma membrane and that will not divide in vivo; and that “cells in a state of no growth” means live cells that will not divide in vivo. Conventional methods of suspending cells in a state of no growth are known to skilled artisans and may be useful in the present invention. For example, cells may be irradiated prior to use such that they do not multiply. Tumor cells may be irradiated to receive a dose of 2500 cGy to prevent the cells from multiplying after administration. Alternatively, ethanol treatment may result in dead cells.

The tumor cells can be prepared from virtually any type of tumor. The present invention contemplates the use of tumor cells from solid tumors, including carcinomas; and non-solid tumors, including hematologic malignancies. Examples of solid tumors from which tumor cells can be derived include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. Hematologic malignancies include leukemias, lymphomas, and multiple myelomas. The following are non-limiting preferred examples of tumor cells to be preserved according to the present invention: melanoma, including stage-4 melanoma; ovarian, including advanced ovarian; small cell lung cancer; leukemia, including and not limited to acute myelogenous leukemia; colon, including colon metastasized to liver; rectal, colorectal, breast, lung, kidney, and prostate cancer cells.

Tumor cell vaccines can be prepared from any of the tumor cell types listed above. Such tumor cell vaccines can comprise preserved cells, i.e., cells treated with ethanol according to the method of the invention. Preferably, the vaccine comprises the same type of cells as the tumor to be treated. Most preferably, the tumor cells are autologous, derived from the patient for whom treatment with the vaccine is intended. Vaccines comprising tumor cells prepared using the method of the invention can used for treatment of both solid and non-solid tumors, as exemplified above. Thus, the invention includes “preserved” vaccines prepared from, and intended for treatment of, solid tumors, including carcinomas; and non-solid tumors, including hematologic malignancies. Preferred tumor types for vaccines include melanoma, ovarian cancer, colon cancer, and small cell lung cancer.

The tumor cells are preferably of the same type as, most preferably syngeneic (e.g., autologous or tissue-type matched) to, the cancer which is to be treated. For purposes of the present invention, syngeneic refers to tumor cells that are closely enough related genetically that the immune system of the intended recipient will recognize the cells as “self”, e.g., the cells express the same or almost the same complement of HLA molecules. Another term for this is “tissue-type matched. ” For example, genetic identity may be determined with respect to antigens or immunological reactions, and any other methods known in the art. Preferably the cells originate from the type of cancer which is to be treated, and more preferably, from the same patient who is to be treated. The tumor cells can be, although not limited to, autologous cells dissociated from biopsy or surgical resection specimens, or from tissue culture of such cells. Nonetheless, allogeneic cells and stem cells are also within the scope of the present invention.

Tumor cells for use in the present invention may be prepared as follows. Tumors are processed as described by Berd et al. (Cancer Res. 1986;46:2572; see also U.S. Pat. No. 5,290,551; U.S. patent application Ser. No. 08/203,004, U.S. patent application Ser. No. 08/475,016, and U.S. patent application Ser. No. 08/899,905). The cells are extracted by dissociation, such as by enzymatic dissociation with collagenase, or, alternatively, DNase, or by mechanical dissociation such as with a blender, teasing with tweezers, mortar and pestle, cutting into small pieces using a scalpel blade, and the like. Mechanically dissociated cells can be further treated with enzymes as set forth above to prepare a single cell suspension.

Tumor cells may also be prepared according to Hanna et al., U.S. Pat. No. 5,484,596. Briefly, tumor tissue is obtained from patients suffering from the particular solid cancer from which the vaccine is to be prepared. The tumor tissue is surgically removed from the patient, separated from any non-tumor tissue, and cut into small pieces, e.g., fragments 2-3 mm in diameter. The tumor fragments are then digested to free individual tumor cells by incubation in an enzyme solution. After digestion, the cells are pooled and counted, and cell viability is assessed. If desired, a Trypan Blue exclusion test can be used to assess cell viability.

In addition, tumor cells can be prepared according to the following procedure (see Hanna et al., U.S. Pat. No. 5,484,596). The tissue dissociation procedure of Peters et al. (Cancer Research 1979;39:1353-1360) can be employed using sterile techniques throughout under a laminar flow hood. Tumor tissue can be rinsed three times in the centrifuge tube with HBSS and gentamicin and transferred to a petri dish on ice. Scalpel dissection removed extraneous tissue and the tumor are minced into pieces approximately 2 to 3 mm in diameter. Tissue fragments are placed in a 75 ml flask with 20-40 ml of 0.14% (200 units/mil) Collagenase Type 1 (Sigma C-0130) and 0.1% (500 Kunitz units/ml) deoxyribonuclease type 1 (Sigma D-0876) (DNAase 1, Sigma D-0876) prewarmed to 37° C. Flasks are placed in a 37° C. water bath with submersible magnetic stirrers at a speed which cause tumbling, but not foaming. After a 30-minute incubation, free cells are decanted through three layers of sterile medium-wet nylon mesh (166t: Martin Supply Co., Baltimore, Md.) into a 50 ml centrifuge tube. The cells are centrifuged at 1200 rpm (250×g) in a refrigerated centrifuge for 10 minutes. The supernatant is poured off and the cells are resuspended in 5-10 ml of DNAase (0.1% in HBSS) and held at 37° C. for 5-10 minutes. The tube is filled with HBSS, washed by centrifugation, resuspended to 15 ml in HBSS and held on ice. The procedure is repeated until sufficient cells are obtained, usually three times for tumor cells. Cells from the different digests are then pooled, counted. Optionally, although not necessarily, cell viability is assessed by the Trypan Blue exclusion test.

Tumor cells, prior to or after ethanol-treatment, can be frozen if stored for extended persiods of time. The cells may be frozen or cryopreserved according to any method known in the art, either before or after any modification to the cells (e.g., haptenization, lysis, etc.) has been made. For example, the dissociated cells may be stored frozen in a freezing medium (e.g., prepared from a sterile-filtered solution of 50 ml Human Serum Albumin [American Red Cross] added to 450 ml of RPMI 1640 (Mediatech) supplemented with L-glutamine and brought to an appropriate pH with NaOH), such as in a controlled rate freezer or in liquid nitrogen until needed. The cells are ready for use upon thawing. Preferably, the cells are thawed shortly before use, or stored for no more than a couple of days before use. Optionally, the cells may be washed once or twice, and then suspended in HBSS without phenol red.

Alternatively, the concentration of dissociated tumor cells can be adjusted to about 5-10×10⁷/ml, or to about 5×10⁷ or 10×10⁷ cells per ml, in HBSS and/or a freezing medium. The freezing medium can be a plain cell growth medium such as HBSS, or a medium or buffer complemented with HSA, sucrose, dextran, or mixtures thereof. Preferably, the freezing medium is based on HBSS and complemented with either HSA/sucrose or HSA/dextran. The cells can also be added in equal volume to chilled 2×freezing medium containing 15% dimethylsulfoxide (DMSO) and 4% human serum albumin (HSA), with or without a suitable concentration of sucrose or dextran. The final suspension of 2 to 4×10⁷ cells/ml is placed in 1.2 ml Nunc freezer vials. In preparation for freezing, the Nunc vials are transferred on ice to a Cryo-Med model 990 Biological Freezer with a model 700 Controller and a model 500 Temperature Recorder for controlled-rate freezing. Care should be taken that the temperature of the individual vials, including the monitor vial, is uniform at the beginning of the freezing process. Vials are cooled at a controlled rate of −1° C./min to a final temperature of −80° C. The vials are then transferred in liquid nitrogen to liquid nitrogen storage. Suitable HSA preparations are available commercially, from, e.g., Baxter Corp. Mississauga, Canada).

An alternative freezing medium is a medium containing 7% sucrose and 10% HSA in HBSS. The cells are stored overnight at −86° C., and then transferred to liquid nitrogen.

### Haptens

In one embodiment, the tumor cells are haptenized. For purposes of the present invention, virtually any small protein or other small molecule that fails to induce an immune response when administered alone, may function as a hapten. A variety of haptens of quite different chemical structure have been shown to induce similar types of immune responses, e.g., TNP (Kempkes et al., J. Immunol., 147:2467, 1991); phosphorylcholine (Jang et al., Eur. J. Immunol., 21:1303, 1991); nickel (Pistoor et al., J. Invest. Dermatol., 105:92, 1995); and arsenate (Nalefski and Rao, J. Immunol., 150:3806, 1993). Conjugation of a hapten to a cell to elicit an immune response may preferably be accomplished by conjugation via ε-amino groups of lysine or —COOH groups. This group of haptens include a number of chemically diverse compounds: dinitrophenyl, trinitrophenyl, N-iodoacetyl-N′-(5-sulfonic 1-naphthyl) ethylene diamine, trinitrobenzenesulfonic acid, dinitrobenzene sulfonic acid, fluorescein isothiocyanate, arsenic acid benzene isothiocyanate, and dinitrobenzene-S-mustard (Nahas and Leskowitz, Cellular Immunol., 54:241, 1980). Once armed with the present disclosure, skilled artisans would be able to choose haptens for use in the present invention.

### Hapenization

When using haptenized cells in the tumor cell composition, modification of the prepared cells with a hapten may be performed by known methods, e.g. by the method of Miller and Clanian (J. Immunol. 1976;117:151). The described procedure involves a 30 minute incubation of tumor cells with DNFB under sterile conditions, followed by washing with sterile saline or Hanks/HSA. Haptenization is also described in the Examples (see below). Other procedures for haptenization are known in the art (see, e.g., International Patent Publications WO 96/40173, WO 00/09140, WO 00/31542, WO 99/56773, WO 99/52546, WO 99/40925, WO 98/14206, WO 00/295, all by Berd et al., and U.S. Pat. No. 5,290,551 to Berd, hereby incorporated by reference in its entirety).

For example, the following procedure may be used for tumor cell haptenization. About 100 mg of DNFB (Sigma Chemical Co., St. Louis, Mo.) is dissolved in about 0.5 ml of 70% ethanol. About 99.5 ml of PBS is added. The solution is stirred overnight in a 37° C. water bath. The shelf life of the solution is about 4 weeks. The cells are thawed and the pellet resuspended in 5×10⁶ cells/ml in Hanks balanced salt solution. About 0.1 ml DNFB solution is added to each ml of cells and incubated for about 30 minutes at room temperature. Similarly, other haptens such as and not limited to trinitrophenyl, N-iodoacetyl-N′-(5-sulfonic 1-naphthyl) ethylene diamine, trinitrobenzenesulfonic acid, fluorescein isothiocyanate, arsenic acid benzene isothiocyanate, trinitrobenzenesulfonic acid, sulfanilic acid, arsanilic acid, dinitrobenzene-S-mustard and combinations thereof may be used.

The tumor cells can also be dual-haptenized, i.e., the same tumor cell preparation can be conjugated with two different haptens. The haptens may comprise reactive groups that react with different functional groups on the tumor cell, such as different amino acids. Such dual-haptenization is described in WO 00/38710 by Berd et al.

Alternatively, the tumor cell can be bi-haptenized or mixed haptenized, i.e., two or more aliquots of a single tumor cell preparation is each coupled to a different hapten, or the same hapten is coupled to different functional groups, can be mixed prior to administration, or administered in conjunction with each other. Bi-haptenization may be conducted as described in the Examples.

Optionally, tumor cells can be frozen before or after haptenization, as described above.

### Formulations

The tumor cells treated with ethanol or another permeabilizing agent or step according to the invention may be included in various formulations. For example, tumor cells may, in haptenized or unmodified form, be useful for preparing tumor vaccines. The different components of such a formulation may be mixed together, and then added to tumor cells. It is also possible to mix one or several of the components with the tumor cells and then to add the remaining component(s). The preparation of the formulation and its addition of the tumor cells are preferably performed under sterile conditions. Preferably, the tumor cells are subjected to ethanol or other treatment before the final formulation. However, one or more components to be included in the final formulation may also be present before or during the treatment step.

The respective proportions of the components of the media according to the invention may be adapted by persons skilled in the art. As illustrated in the Examples, the proportions may be modified although certain concentration ranges are preferred.

Generally, an appropriate buffered medium is used for tumor cell formulation. In its essence, a buffered medium is an isotonic buffered aqueous solution, such as phosphate buffered saline (PBS), Tris-buffered saline, or HEPES buffered saline. In a preferred embodiment, the medium is a buffered cell culture medium such as plain Hank's medium (not containing phenol red), e.g., as sold commercially by Sigma Chemical Co. (St. Louis, Mo., USA). Other tissue culture media can also be used, including basal medium Eagle (with either Earle's or Hank's salts), Dulbecco's modified, Eagle's medium (DMEM), Iscove's modified Dulbecco's medium (IMDM), Medium 199, Minimal Essential Medium (MEM) Eagle (with Earle's or Hank's salts), RPMI, Dulbecco's phosphate buffered salts, Earle's balanced salts (EBSS), and Hank's Balanced Salts (HBSS). These media can be supplemented, e.g., with glucose, Ham's nutrients, or HEPES. Other components, such as sodium bicarbonate and L-glutamine, can be specifically included or omitted. Media, salts, and other reagents can be purchased from numerous sources, including Sigma, Gibco, BRL, Mediatech, and other companies.

Generally, human serum albumin (HSA) is also included, as described below. In addition, a composition or formulation of the invention may contain components in addition to HSA to further stabilize the haptenized tumor cells. Examples of such components include, but are not limited to, carbohydrates and sugars such as dextrose, sucrose, glucose, and the like, e.g., at a 5% concentration; medium to long chain polyols such as glycerol, polyethylene glycol, and the like, e.g., at 10% concentration; other proteins; amino acids; nucleic acids; chelators; proteolysis inhibitors; preservatives; and other components. Preferably, any such constituent of a composition of the invention is pharmaceutically acceptable.

### Human Serum Albumin

In a preferred embodiment, the tumor cell formulations of the invention comprise a concentration or amount of a protein such as, e.g., albumin, which is effective to stabilize the tumor cells. An amount of protein effective to stabilize the tumor cells may be added before and/or after ethanol treatment, or, in the case of haptenized tumor cells, before and/or after haptenization. In a preferred embodiment, the albumin is human serum albumin or HSA. HSA has been shown to stabilize solutions of proteins, including protein antigens, and small organic molecules such as hemin (Paige, A. G. et al., Pharmaceutical Res., 12:1883-1888, 1995; Chang, A. -C. and R. K. Gupta, J., Pharm. Sci., 85:129-132, 1996; Niemeijer, N. R. et al., Ann. Allergy Asthma Immunol., 76:535-540, 1996; and Cannon, J. B. et al., PDA:J. Pharm. Sci. & Tech., 49:77-82, 1995), as well as haptenized tumor cell compositions (see WO 00/29554, corresponding to U.S. Pat. No. 6,248,585).

The HSA used within the framework of the present invention may be either of natural origin (purified HSA) or of recombinant origin (rHSA). Naturally, for delivery of a formulation in vivo, it is preferable to use an autologous or non-immunogenic serum albumin. Thus, for human therapy, HSA is desirable and preferred. However, the skilled person can immediately appreciate that any serum albumin can be used in the practice of this invention, and, more particularly, any autologous serum albumin can be used in connection with tumor cell vaccine for cancer treatment in any non-human animal as well. In a specific embodiment, a Human Serum Albumin Solution (American Red Cross), which is a 25% HSA solution, is used.

Advantageously, a recombinant or natural HSA is used which meets certain quality criteria (e.g., homogenetic, purity, stability). Thus, the pharmacopoeias set a number of parameters for the albumin solutions, namely a pH value, a protein content, a polymer and aggregate content, an alkaline phosphatase content, and a certain protein composition. It imposes, furthermore, a certain absorbance, the compliance with tests for sterility, pyrogens, and toxicity (see “Albumini humai solutio”, European Pharmacocpoeia (1984), 255). The use of an albumin composition corresponding to these criteria, although not essential, is particularly preferred.

Generally, the HSA formulation of the invention is made by adding HSA powder or solution to the selected culture medium/balanced salt solution, to achieve the desired final concentration. The final concentration of HSA is preferably, in weight to volume, from about 0.1% to 10%, even more preferably from about 0.25% to about 2%, and most preferably about 1%.

Additional information about the use of albumin in formulations of tumor cells, especially haptenized tumor cells, can be found in WO 00/29554, corresponding to U.S. Pat. No. 6,248,585 .

### Vaccine Preparation and Administration

The compositions of the invention may be administered in a mixture with a pharmaceutically-acceptable carrier, selected with regard to the intended route of administration and standard pharmaceutical practice. Dosages may be set with regard to weight and clinical condition of the patient. The proportional ratio of active ingredient to carrier naturally depends on the chemical nature, solubility, and stability of the compositions, as well as the dosage contemplated. The amounts to be used of the tumor cells of the invention depend on such factors as the affinity of the compound for cancerous cells, the amount of cancerous cells present and the solubility of the composition. The compounds of the present invention may be administered by any suitable route, including inoculation and injection, for example, intradermal, intravenous, intraperitoneal, intramuscular, and subcutaneous. For example, the composition may be administered by intradermal injection into 3 contiguous sites per administration on the upper arms or legs, excluding limbs ipsilateral to a lymph node dissection.

### Tumor Cell Dose

A predetermined number or concentration of cells is included in each vaccine dose. To prepare the vaccine dosage forms to contain the right number and/or concentration of cells, the cells in a tumor cell preparation can be counted by any suitable method known in the art. For example, cells can be counted manually using a microscope and standard cell counting chambers, or by using automatic cell counters such as, e.g., Beckman Coulter cell counters. Since the method does not require distinguishing between live and “dead” cells, and in some embodiments, even prefer “dead cells”, Trypan Blue and other means which are selective for live or dead cells can be omitted. The concentration of cells can then be adjusted by diluting the cells with a sterile solution so that a certain volume corresponds to the number of cells to be injected into the patient, and this volume aliquoted into storage vials.

In one embodiment of the invention, the composition comprises a vaccine comprising about 10×10⁴ to 1×10⁸, more preferably 1×10⁶ to about 25×10⁶, even more preferably about 2.5×10⁶ to about 7.5×10⁶, tumor cells suspended in a pharmaceutically acceptable carrier or diluent, such as, but not limited to, Hank's solution (HBSS), saline, phosphate-buffered saline, and water. In another embodiment, the tumor cell vaccine comprises from about 5×10⁴ to about 5×10⁶ cells, for example; 5×10⁴, 5×10⁵, or 5×10⁶ tumor cells. Preferably, the tumor cells are dead and do not exclude Trypan Blue or another supravital dye.

### Adjuvants

In preferred embodiment, a tumor cell composition may be administered with an immunological adjuvant. While the commercial availability of pharmaceutically acceptable adjuvants is limited, representative examples of adjuvants include Bacille Calmette-Guerin, BCG, or the synthetic adjuvant, QS-21 comprising a homogeneous saponin purified from the bark of Quillaja saponaria, Corynebacterium parvum, (McCune et al., Cancer 1979 ;43:1619), and IL-12.

It will be understood that the adjuvant is subject to optimization. In other words, the skilled artisan can engage in no more than routine experimentation and determine the best adjuvant to use.

### Immunostimulants and Combination Therapies

The tumor cell compositions may be co-administered with other compounds including but not limited to cytokines such as interleukin-2, interleukin-4, gamma interferon, interleukin-12, GM-CSF. The tumor cells preparations of the invention may also be used in conjunction with other cancer treatments including but not limited to chemotherapy, radiation, antibodies, antisense oligonucleotides, and gene therapy. In a preferred embodiment, cyclophosphamide is used as adjunctive chemotherapy in treatment regimes involving the present tumor cell vaccines.

### EXAMPLES

The following examples are illustrative of the invention, but not limiting thereof.

### Example 1

### Ethanol Treatment of Mixed-Haptenized Melanoma Cells

This Example describes a strategy for preparation of a bi-haptenized vaccine, i.e., haptenization of two different tumor cell preparations with two different haptens, followed by ethanol treatment to preserve the cells. One tumor cell preparation was modified with dinitrophenyl (“DNP”) while the other tumor cell preparation was modified with sulfanilic acid “SA”).

Materials

Wash and Thaw solution:

500 ml Hanks (HBSS, Sigma catalogue # 21-022-CV)

Add 0.5 g EDTA (Sigma catalogue # E-5134)

Adjust pH to 7.2 with 5 N NaOH

Add 2.0 ml HSA (as 25% solution (final concentration=0.1%)).

Sterile filter through 0.2μ filter into sterile plastic bottle attached to filtration unit (Nalgene—Fisher catalog # 09-740-25A)

Shelf life=30 days—store at 4° C.

Thawing

Thaw cells rapidly in water bath. Remove before last ice crystal has melted. Dilute DMSO in Wash & Thaw solution, as follows: For each ml cells, add 0.05 ml and swirl for 30 sec, then add 0.1 ml and swirl for 30 sec, then add 0.2 ml and swirl for 30 sec, then add 0.4 ml and swirl for 30 sec, then add 0.8 ml and swirl for 30 sec. Allow cells to sit at room temperature for 5 minutes. Add 10 ml Wash & Thaw solution. Spin at 1100 RPM for 7 minutes. Aspirate supernatant and suspend pellet in 10 ml Hanks Buffered Saline Solution (HBSS) without albumin. Spin at 1100 RPM for 7 minutes. Aspirate supernatant and suspend pellet in 2. ml HBSS without albumin. Do cell count as per Cell Counting Procedure (below). Then divide cell suspension into two 1 ml aliquots. Label one tube “DNP” and the other “SA”. Place the “SA” tube at 4° C.

Cell Counting Procedure

1) Resuspend pellet in 2.0 ml Hanks

2) Remove 25 μl of cell suspension using Eppendorf pipettor with sterile tip extension. Add to 0.2 ml of Hanks solution; then add 25 μl of Trypan Blue solution

3) Mix with Pasteur pipette and apply to hemacytometer

4) Count cells belonging to the following categories: a) large, Trypan-Blue (−); b) small, trypan-blue (−); c) dead, trypan blue (+). Count a minimum of 40 (and a maximum of 100) large trypan-blue (−) cells. Count at least a portion of two large squares (there are 9 large squares in the hemacytometer). It may be necessary to count all 9 squares to reach the minimum count of 40 large cells. If there are <40 large cells in the 9 squares, it is necessary to re-pellet the cell suspension and follow procedure B (see below)

B—If Number Live Tumor Cells Originally Frozen is <5×10⁶ per Vial

1) Resuspend pellet in 0.5 ml Hanks

2) Add 25 μl of cell suspension to 0.2 ml of Hanks solution; then add 25 μl of trypan blue solution.

3) Mix with Pasteur pipette and apply to hemacytometer

4) Count cells—a) large, trypan-blue (−); b) small, trypan-blue (−); c) dead, trypan blue (+). Count a minimum of 40 (and a maximum of 100) large trypan-blue (−) cells. Count at least a portion of two large squares (there are 9 large squares in the hemacytometer). If there are <40 large cells in all 9 squares, use the count, but make a control count.

Calculations

The total number of cells (×10⁶)=(C×V×10 )/(S×100)

C=No. cells counted

V=(volume of suspension)=2.0 or 0.5

S=No. large squares counted

DNP Modification

To the “DNP” tube add HBSS without albumin to bring the concentration of cells (intact tumor cells (TC)+lymphocytes (LY)+dead cells) to 5×10⁶/ml. For each 1.0 ml of cell suspension, add 0.1 ml of DNFB solution. Mix and incubate at room temperature for 30 minutes; gently mix every 10 minutes.

SA Modification

Reagents for SA Modification:

Hanks Balanced Salt Solution (HBSS)

10% sodium nitrite: 10 g sodium nitrite (Sigma S-3421 powder)+100 ml water; sterile filter through 0.2μ membrane; keep for 1 month.

0.1N hydrochloric acid—buy as Sigma 210-4 (endotoxin-free)

Sulfanilic acid: add 100 mg sulfanilic acid (Sigma—S-5643 (100 g) (anhydrous)) to 10 ml 0.1N hydrochloric acid

SA diazonium salt: add ice-cold sodium nitrite dropwise to sulfanilic acid—stir for 30 sec after each drop, then add droplet to starch-iodide paper until blue color appears (about 15 drops)—then stop (the final concentration of sulfanilic acid diazonium salt should be 40 mM). Sterile filter the sulfanilic acid diazonium salt through 0.2 u membrane and store at 4° C. for no more than 7 days.

While the DNP cells are incubating, dilute the SA diazonium salt 1:8 in Hanks without albumin, and adjust the pH to 7.2 by dropwise addition of 1N NaOH (2-3 drops). Sterile filter the solution through 0.2μ membrane. Pellet the “SA” tube by centrifuging at 1100 RPM for 7 minutes. Aspirate supernatant. Add a quantity of the diluted diazonium salt to the pellet to make a cell concentration (intact TC+LY+dead) of 5×10⁶/ml. Immediately resuspend. Incubate for 5 minutes at room temperature.

As soon as the DNP and SA modifications are finished (30 minutes and 5 minutes, respectively), stop the reactions by adding 0.5 ml of the stock solution of human serum albumin (25% solution) to the tube, capping, and mixing. Pellet the cells by spinning at 1100 RPM for 7 minutes. Wash the cells twice in HBSS+1.0% HSA.

Ethanol Treatment

After the last centrifugation, resuspend the cells in the DNP and SA tubes in 1 ml cold (4° C.) HBSS with 1% HSA. Place the tubes on ice (4° C.). Add 3 ml of ice-cold 50% ethanol to each tube while vortexing at low speed. Incubate the tubes at 4° C. for 10 minutes. Pellet cells by spinning at 1100 RPM for 7 minutes. Aspirate supernatant, resuspend in 10 ml HBSS+1% HSA, and pellet by spinning at 1100 RPM for 7 minutes. Aspirate supernatant and resuspend in 2. ml Hanks+1% HSA. Other ethanol concentrations may be used in this procedure, for example by varying either the ethanol concentration in the stock solution added to the HBSS solution, and/or by varying the amount of ethanol added to the HBSS solution.

Perform cell count of SA and DNP tubes. Addition of Trypan Blue is not necessary (the cells are fixed and all will take up Trypan Blue). Count only large cells (tumor cells) and small cells (lymphocytes). No discrimination is made between live tumor cells and dead ones (most if not all cells ate dead). Add a quantity of HBSS+1% HSA to each tube to make the cell concentration (large cells only) to 1×10⁶/ml. Mix the DNP and SA tubes by adding to a third tube labeled “BIHAP” as follows.

| Vaccine Dose | Volume of DNP Cells | Volume of SA Cells |
|---|---|---|
| 10 × 10⁶ | 5 ml | 5 ml |
| 5 × 10⁶ | 2.5 ml | 2.5 ml |
| 2.5 × 10⁶ | 1.25 ml | 1.25 ml |
| 1.25 × 10⁶ | 0.625 ml | 0.625 ml |

Pellet the BIHAP tube by spinning at 1100 RPM for 7 minutes. Aspirate supernatant and resuspend in 0.15 ml HBSS+1.0% HSA. Place suspension into a properly labeled cryotube: a) patient's name; b) patient study number; and c) date when cells were cryopreserved. Keep the vaccine at 4° C. until administered.

### Example 2

### Optimization of Ethanol Concentration

This Example describes experiments in which the mixed-haptenized tumor cell retention of the HLA class I antigen was measured, by flow cytometry, after treatment with different ethanol concentrations. Mixed-haptenized cells were prepared as described in Example 1. Ethanol treatment of the mixed-haptenized cells was investigated in order to produce a vaccine that was stable enough to allow time for quality control testing and for shipping, while retaining the antigenicity of the vaccine. Ethanol is known to be an excellent cell fixative, but high concentrations can diminish the availability of cell surface antigens that can be important to the effectiveness of a vaccine.

Ethanol treatment was performed as follows. After the last centrifugation, resuspend the cells in the DNP and SA tubes in 1 ml cold (4° C.) Hanks with 1% HSA. Place the tubes on ice (4° C.). Add 3 ml of ice-cold ethanol to each tube while vortexing at low speed. Incubate the tubes at 4° C. for 10 minutes. Pellet cells by spinning at 1100 RPM for 7 minutes. Aspirate supernatant, resuspend in 10 ml Hanks+1% HSA, and pellet by spinning at 1100 RPM for 7 minutes. Aspirate supernatant and resuspend in 2 ml Hanks+1% HSA.

Flow Cytometry

Flow cytometry analysis was conducted as follows: Aliquot cells in 10×75 mm tubes, pellet, and resuspend in 50 μl Hanks+HSA. Add a predetermined optimum concentration of each antibody in a volume of 10-50 μl. Vortex the tubes and incubate for 30 minutes at 4° C. Washed the cells twice in 2 ml Hanks+HSA, pellet, and resuspend in 500 μl Hanks+HSA. Maintain cells at 4° C. until analysis. The analysis can be performed with a Coulter EPICS XL flow cytometer. Forward light scatter gates are set to include cells and to exclude debris. The percentage of cells binding various antibodies iss determined by the percentage positive in the green fluorescence channel.

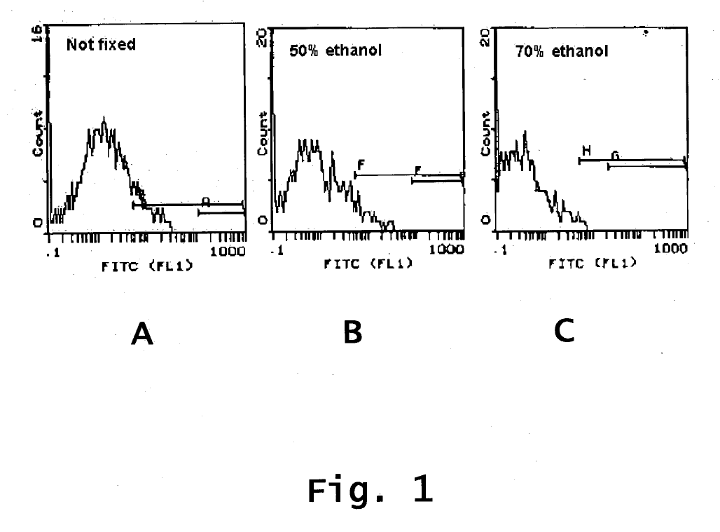

The flow cytometry histograms in FIG. 1 indicated that the cell-associated presence of the invariant region of HLA class I (detected by the monoclonal antibody W6/32) was greatly reduced by fixation with 70% ethanol, i.e., 3 ml 70% ethanol mixed with 1 ml mixed-haptenized cell suspension (right panel). 100% ethanol reduced class I display even further. However, reduction of the ethanol concentration to 50% (middle panel) preserved the cellular display of class I. Therefore, 50% ethanol (final concentration=38%) was chosen as optimal concentration.

### Example 3

### Retention of HLA Class I Antigen After Ethanol Treatment

This Examples describes the cell recovery and antigenicity of haptenized cells when stored. Cell counting and flow cytometry was conducted as described in Examples 1 and 2.

As expected and as shown in the table below, bihaptenization followed by ethanol treatment resulted in loss of melanoma cells. However, the remaining cells appeared intact by microscopic examination and flow cytometry (see TABLE 1).

TABLE 1
Yield of Melanoma Cells (live + dead) Following Hapten Modification
and Ethanol Treatment.

| Patient | Hapten | Post-Thaw* | Post-Haptenization + Fixation* | Yield |
|---|---|---|---|---|
| 1 | DNP | 18.7 | 9.8 | 52% |
| 1 | SA | 18.7 | 8.8 | 47% |
| 2 | DNP | 23.1 | 4.2 | 18% |
| 2 | SA | 23.1 | 2.8 | 12% |
| 3 | DNP | 29.0 | 11.4 | 39% |
| 3 | SA | 29.0 | 10.6 | 37% |
| 4 | DNP | 11.9 | 3.2 | 27% |
| 4 | SA | 11.9 | 3.0 | 25% |
| 5 | DNP | 8.8 | 5.6 | 64% |
| 5 | SA | 8.8 | 3.2 | 36% |

*No. cells × 10⁶

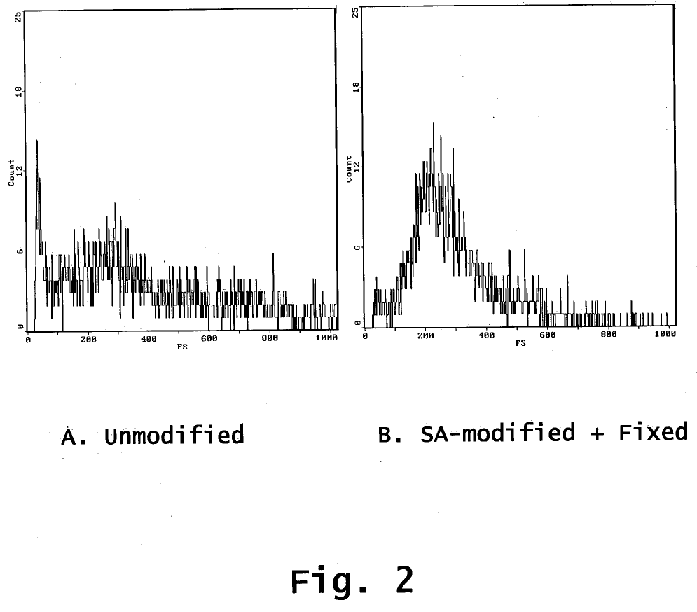

Flow cytometric analysis of mixed-haptenized, ethanol-treated melanoma cells showed a consistent change in forward light scatter: the peak was more clearly defined and shifted to the left, as shown in the histograms in FIG. 2. This is an indication of fixation, which causes a characteristic shift in the forward light scatter peak.

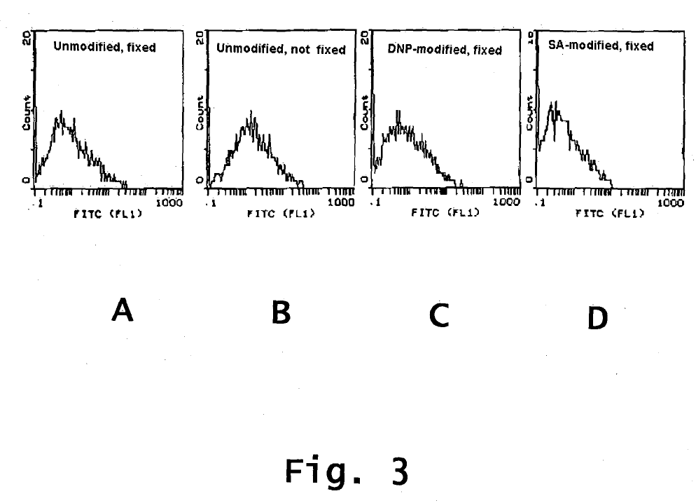

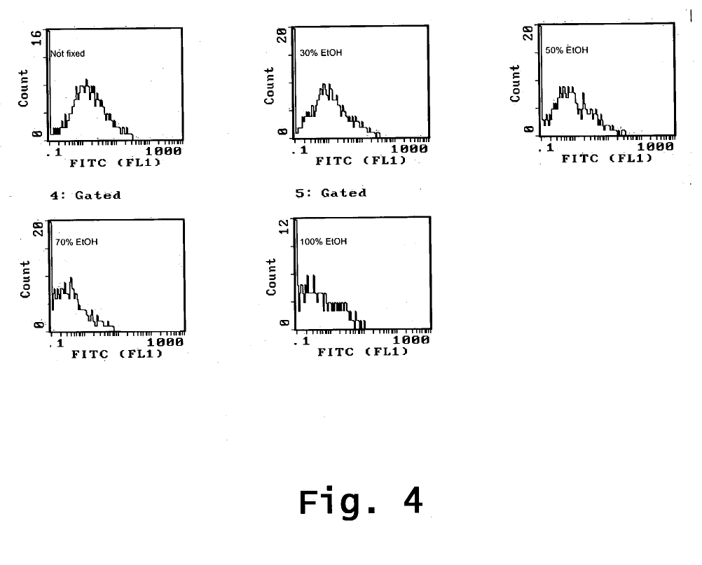

Since all the mixed-haptenized, ethanol-treated cells were dead, as assessed by uptake of a supravital dye (trypan blue), it was important to demonstrate that they retained display of surface antigens. The histograms in FIG. 3 show that cell-association with surface class I antigen (detected by antibody W6/32) was intact and only slightly diminished compared with unmodified and/or non-ethanol-treated melanoma cells. FIG. 4 shows a comparison between (non-haptenized) unfixed cells, and cells treated with 30%, 50%, 70%, and 100% ethanol.

### Example 4

### Stability of Ethanol-Treated Cells

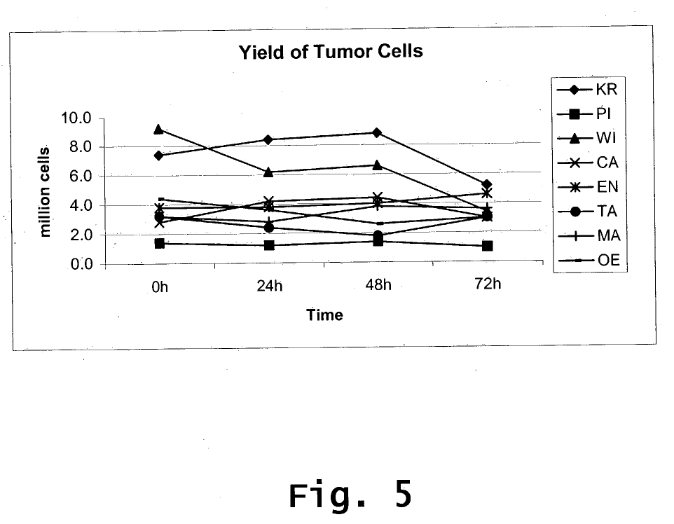

As expected, mixed-haptenized, ethanol-fixed melanoma cells were much more stable than mixed-haptenized unfixed cells, of which 90% were lost after 4 hours at 40° C. TABLE 2 and FIG. 5 show that these fixed cells could be stored for 48-72 hours at 4° C. in Hanks+1% human serum albumin with minimal loss of tumor cells.

TABLE 2
Short-Term Stability of Mixed-Haptenized, Ethanol-Treated Cells.

| Sample No. | 0 h* | 24 h* | 48 h* | 72 h* |
|---|---|---|---|---|
| 1 | 7.4 | 8.4 | 8.8 | 5.2 |
| 2 | 1.4 | 1.2 | 1.4 | 1.0 |
| 3 | 9.2 | 6.2 | 6.6 | 3.4 |
| 4 | 2.8 | 4.2 | 4.4 | 3.0 |
| 5 | 3.8 | 3.8 | 4.0 | 4.6 |
| 6 | 3.2 | 2.4 | 1.8 | 3.0 |
| 7 | 3.2 | 2.8 | 3.8 | 3.6 |
| 8 | 4.4 | 3.6 | 2.6 | 3.0 |

*No. cells × 10⁶

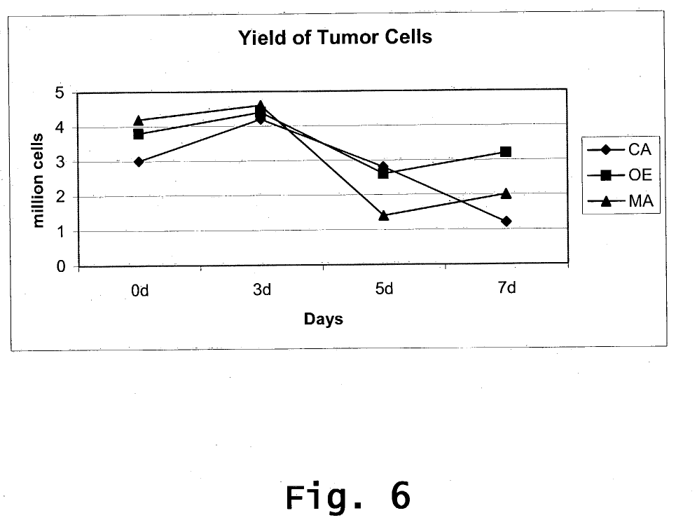

Longer term studies, shown in TABLE 3 and FIG. 6, indicated that the loss of tumor cells was significant only after 5 or 7 days at 40° C., although even at 7 days the recovery average was 57%. From the data in TABLE 2, it was found that, in average, 95%, 98%, and 85% of the cells remained after 24 h, 48 h, and 72 h of incubation, respectively.

TABLE 3
Long-Term Stability of Mixed-Haptenized, Ethanol-Treated Cells.

| Sample No. | 0 d* | 3 d* | 5 d* | 7 d* |
|---|---|---|---|---|
| 9 | 3 | 4.2 | 2.8 | 1.2 |
| 10 | 3.8 | 4.4 | 2.6 | 3.2 |
| 11 | 4.2 | 4.6 | 1.4 | 2 |

*No. cells × 10⁶

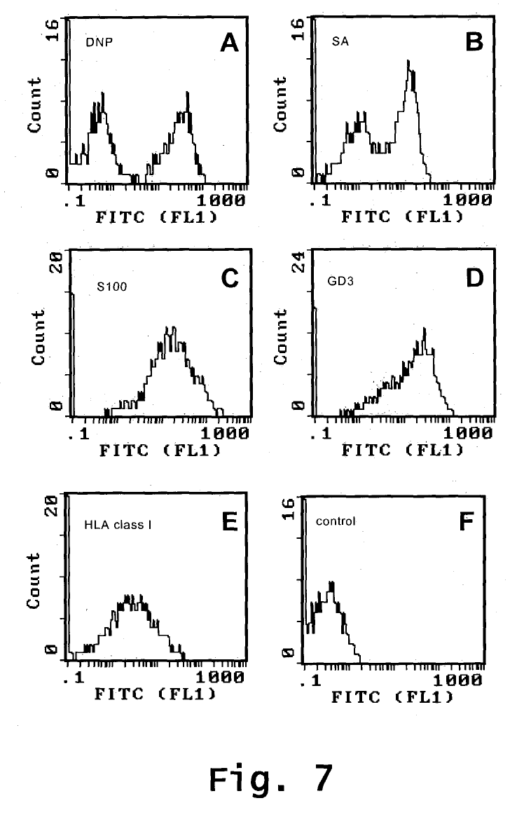

Mixed-haptenized, fixed vaccine stored for 3 days retained their display of antigens and hapten modification. The three sets of histograms in FIG. 7 show stability of cell-associated HLA class I and the melanoma-associated antigens S-100 and GD3, and the presence of sulfanilic acid and DNP. Similar results were obtained for the antigens HMB-45 and MART-1. (S100: see Weiss et al., Lab Invest 49:299-308, 1983; HMB-45: see Thomson and Mackie, J Am Acad Dermatol 21:1280-1284, 1989; R24 anti-GD3 antibody: see Houghton et al., Proc. Natl. Acad. Sci. USA 82:1242-1246, 1985; MART-1: see Cole et al., Cancer Res. 54:5265-5268, 1994).

### Example 5

### Inhibition of Proliferation of Mixed-Haptenized and Ethanol-Treated Cells

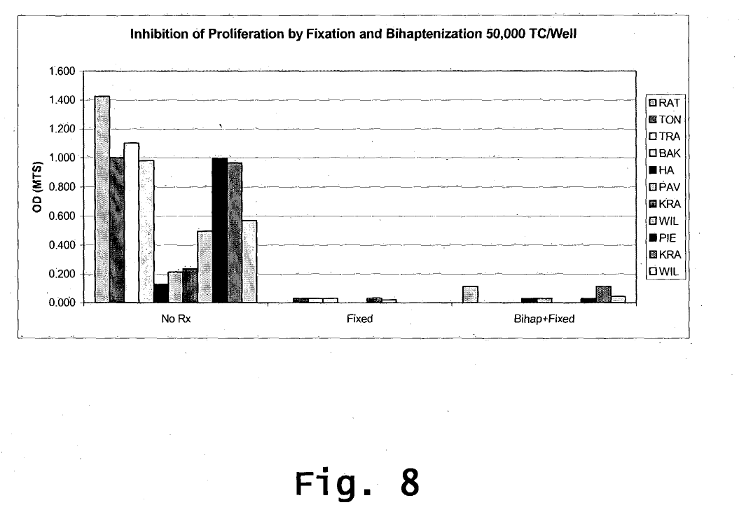

This Example shows that ethanol-treatment produces attenuated or dead cells, i.e., cells incapable of cellular proliferation (FIG. 8). The assay (“MTS Cell Proliferation Assay”) was performed using mixed-haptenized and ethanol-treated cells prepared as described above.

MTS Cell Proliferation Assay

The cell Titer 96 Aqueous Non-Radioactive Cell Proliferation Assay is a colorimetric method for determine the number of viable cells in proliferation or chemosensitivity assays. The Cell Titer 96 Aqueous Assay is composed of solutions of the tetrazolium compound (3-(4,5-dimethylthiazol-2-yl)-5-(3-carboxymethoxyphenyl)-2-(4-sulfophenyl)-2H-tetrazolium, inner salt (MTS) and an electron coupling reagent (phenazine methosulfate; PMS). MTS is bioreduced by cells into a formazan product that is soluble in tissue culture medium. The absorbance of the formazan at 490 nm can be measured directly from 96 well assay plates without additional processing. The conversion of MTS onto aqueous, soluble formazan is accomplished by dehydrogenase enzymes found in metabolically active cells. The quantity of formazan product as measured by the amount of 490 nm is directly proportional to the number of living cells in culture.

Preparation of Media:

Prepare 10% FCS in RPMI+Penicillin/Streptomycin. Mix 10 ml FCS (Fetal Calf Serum) or AB, 1 ml Penicillin-Streptomycin, 1 ml Hepes buffer, 2 ml Glutamine, 1 ml non-essential amino acids, and 85 ml RPMI. Sterile filter through 0.2μ filtration unit.

Preparation of MTS:

1. Thaw MTS solution and PMS solution vials in 37 degrees Celsius water bath.

2. Pipet PMS solution into MTS vial and mix thoroughly.

3. Pipet 2 ml of combined solution into 2 ml cryogenic vials

4. Store at −20 degrees Celsius, and avoid exposure to direct light.

Preparation of Cells:

1. Thaw tumor cell suspensions by SOP.

2. Aliquots of a tumor cell sample may be treated to inhibit replication and/or metabolic activity, e.g., irradiation, haptenization, or ethanol fixation.

3. Suspend cells at 10×10⁶/ml in medium.

4. Place suspension at 4 degrees Celsius until needed.

Preparation of Plates:

1. Label a 96 well plate with patient name and date.

2. Using multi pipette place 100 ul of medium in wells A1-A6 to H1-H6.

3. Using multi pipette place 100 ul aliquots of untreated (viable) cells in wells B1-B3.

4. Using multi pipette place 100 ul aliquots of treated cells in wells B4-B6.

5. Perform a two fold dilution going from wells B1-B6 to H1-H6

6. Incubate plates at 37° C. for required time (48 hours to 4 weeks).

Reading of Plates:

1. At the end of the incubation period, pipet 10 ul of MTS in wells A1-A6 through H1-H6

2. Incubate plates at 37 deg for 3 to 4 hours.

3. Place plates on ELISA plate reader, and record absorbance at 490 nm.

The melanoma cells were not irradiated. As shown in FIG. 8, irradiation is not necessary to abrogate the ability of the melanoma cells to grow, as either fixation in 50% ethanol or fixation preceded by bihaptenization completely inhibited the proliferative capacity of melanoma cells as indicated by incorporation of MTS.

### Example 6

### Elicitation of DTH by DNP-Modified, Ethanol-Treated Cells

Seven patients were immunized with DNP-modified cells according to standard procedures. Five of the patients suffered from melanoma, and two from ovarian carcinoma. The patients were immunized with DNP-modified melanoma or ovarian cells (not fixed) according to established protocols, and underwent post-vaccine DTH testing simultaneously with autologous tumor cells prepared in the standard fashion (i.e., not treated) and the same preparation of cells that had been fixed in 50% ethanol. The cells had been stored for a couple of hours after ethanol treatment. DTH-testing was conducted as follows: Approximately 1×10⁶ tumor cells (with non-fixed cells this was defined as trypan blue-excluding tumor cells; with fixed cells this was defined as all tumor cells—no trypan blue added) was injected intradermally on the patient's forearm. Control material (diluent=Hanks+HSA) was similarly injected. After 48 hours the patient's arm was inspected. For each injection site, the largest diameter of induration was measured (in millimeter) with a ruler.

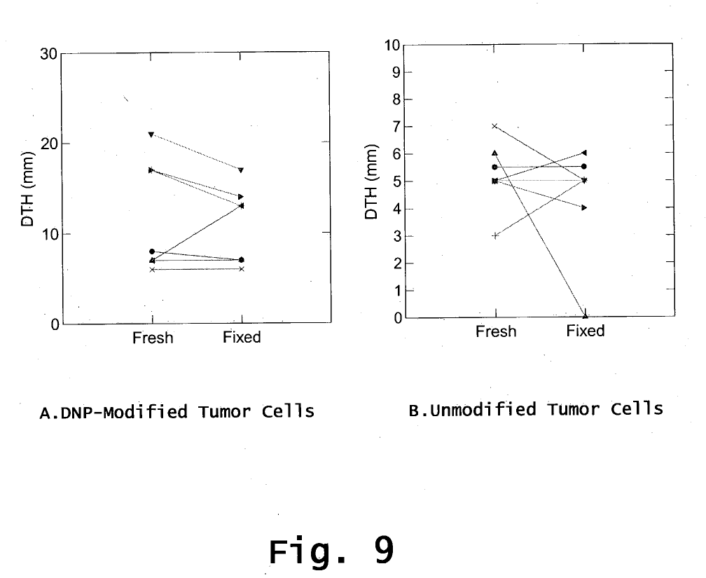

The results are shown in FIGS. 9A and 9B, and in TABLE 4. In the figures, each line represents one patient. Ethanol-fixed cells elicited DTH responses that were indistinguishable from those elicited by non-fixed cells, both for DNP-modified tumor cells and for unmodified cells (p=0.696 and 0.395, respectively).

TABLE 4
DTH-response elicited by Ethanol-Treated Cells.

| | | | | | DTH |
|---|---|---|---|---|---|
| Patient | Histology | Date | Material* | Fixed? | (mm) |
| 1 | Mel | Mar. 20, 2001 | TC UNMOD | No | 5 |
| 1 | Mel | Mar. 20, 2001 | TC UNMOD | Yes | 4 |
| 2 | Mel | Mar. 26, 2001 | TC UNMOD | No | 5 |
| 2 | Mel | Mar. 26, 2001 | TC UNMOD | Yes | 5 |
| 3 | Mel | Apr. 09, 2001 | TC UNMOD | No | 3 |
| 3 | Mel | Apr. 09, 2001 | TC UNMOD | Yes | 5 |
| 4 | Mel | Apr. 23, 2001 | TC UNMOD | No | 6 |
| 4 | Mel | Apr. 23, 2001 | TC UNMOD | Yes | 0 |
| 5 | Mel | Mar. 12, 2001 | TC UNMOD | No | 5 |
| 5 | Mel | Mar. 12, 2001 | TC UNMOD | Yes | 5 |
| 6 | Ov | Mar. 27, 2001 | TC UNMOD | No | 5 |
| 6 | Ov | Mar. 27, 2001 | TC UNMOD | Yes | 6 |
| 7 | Ov | May 01, 2001 | TC UNMOD | No | 7 |
| 7 | Ov | May 01, 2001 | TC UNMOD | Yes | 5 |
| 1 | Mel | Mar. 20, 2001 | TC-DNP | No | 17 |
| 1 | Mel | Mar. 20, 2001 | TC-DNP | Yes | 14 |
| 2 | Mel | Mar. 26, 2001 | TC-DNP | No | 8 |
| 2 | Mel | Mar. 26, 2001 | TC-DNP | Yes | 7 |
| 3 | Mel | Apr. 09, 2001 | TC-DNP | No | 17 |
| 3 | Mel | Apr. 09, 2001 | TC-DNP | Yes | 13 |
| 4 | Mel | Apr. 23, 2001 | TC-DNP | No | 7 |
| 4 | Mel | Apr. 23, 2001 | TC-DNP | Yes | 7 |
| 5 | Mel | Mar. 12, 2001 | TC-DNP | No | 21 |
| 5 | Mel | Mar. 12, 2001 | TC-DNP | Yes | 17 |
| 6 | Ov | Mar. 27, 2001 | TC-DNP | No | 7 |
| 6 | Ov | Mar. 27, 2001 | TC-DNP | Yes | 13 |
| 7 | Ov | May 01, 2001 | TC-DNP | No | 6 |
| 7 | Ov | May 01, 2001 | TC-DNP | Yes | 6 |

*TC UNMOD = Unmodified tumor cells;
TC-DNP = DNP-modified tumor cells.

### Example 7

### Clinical Study with Ethanol-Treated Cells

This Example outlines the design of a clinical study using ethanol-treated cells.

A novel human cancer vaccine, consisting of autologous tumor cells modified with the hapten, dinitrophenyl (DNP), has been developed. The DNP-modified vaccine induces unique immunological effects and shows clinical efficacy. A second-generation vaccine composed of autologous tumor cells, half of which have been modified with DNP and half with a second hapten, sulfanilic acid (SA), has also been developed. Moreover, because the vaccine composition is fixed with a low concentration of ethanol and frozen, it will more readily meet current regulatory requirements.

A phase I trial of the mixed haptenized vaccine in patients with stage IV melanoma is conducted, testing four dosage levels. The major endpoints are the development of delayed-type hypersensitivity (DTH) to DNP-modified, SA-modified, and unmodified autologous tumor cells. Also, the development of tumor inflammatory responses is studied.

Subsequently, a phase II trial using the lowest dose that is found to be immunologically effective in the phase I trial is conducted. The immunological basis of a newly discovered phenomenon—the importance of the timing of a vaccine “induction” dose, is investigated. The hypothesis that the administration of an induction dose timed optimally with administration of low dose cyclophosphamide results in selective depletion of suppressor T cells that would otherwise down-regulate or abrogate the anti-tumor immune response is tested. Peripheral blood lymphocytes are obtained from patients at various time points and assayed for the presence of suppressor cells. It is then determined whether such suppressor cells have a characteristic phenotype, CD4⁺CD25⁺ with co-expression of CTLA4, and whether upon stimulation they produce the immunoregulatory cytokine, IL10. Finally, the ability of the suppressor cells to down-regulate in vitro T cell responses to alloantigens, hapten-modified tumor cells, and unmodified tumor cells, is tested. These studies provide insights into the immunobiology of human cancer vaccines and assist in the development of more effective immunotherapy strategies.

### Example 8

### Clinical Protocol for Mixed-Haptenized, Ethanol-Treated Tumor Cell Vaccine

This Example describes a phase I-II trial of a human cancer vaccine, consisting of cryopreserved, irradiated autologous tumor cells, half of which have been modified with the hapten, dinitrophenyl (DNP) and half of which have been modified with the hapten, sulfanilic acid (SA). The study subjects are patients with stage IV melanoma (non-regional metastases) who have at least one resectable metastasis. The tumor tissue obtained is dissociated into single cell suspensions and cryopreserved. The yield of tumor cells (live+dead) should be ≧100×10⁶. After recovery from surgery, the patients receive a seven-week course of treatment. The DNP-modified and SA-modified cells are mixed in equal numbers, fixed with ethanol, aliquotted, and frozen. The vaccine is administered as follows: a) induction dose day 1, b) low dose cyclophosphamide day 8, c) starting day 11, weekly vaccine mixed with BCG for six weeks, d) booster injection of vaccine mixed with BCG at 6 months. Three dose levels of mixed haptenized vaccine are studied. Low dose cyclophosphamide is administered between the first and second vaccine injections, because of its ability to augment the development of cell-mediated immunity to tumor-associated antigens. The patients are evaluated for delayed-type hypersensitivity (DTH) to autologous tumor cells and for toxicity. The development of tumor inflammation and tumor regression is recorded.

Eligibility

Patients, ages 18 and above, have stage IV melanoma (non-regional metastases) with at least one metastasis that is resectable and an estimated survival of at least 6 months. Patients with residual metastases following surgery as well as those who are clinically tumor-free are included. The mass of excised tumor must be sufficient to obtain ≧100×10⁶ tumor cells (live+dead). Allowable metastatic sites from which tumor may be harvested include: lymph nodes, lung, liver, adrenal, and subcutaneous tissue. Metastatic sites that are not allowed are: bone, brain, or gastrointestinal tract. A sufficient number of vaccine cells have been prepared and frozen to administer a course of therapy, and vaccines must have passed lot release tests.

Surgery and Tumor Acquisition

Patients undergo surgical resection of metastases by standard techniques. The tumor tissue is hand delivered or shipped to the laboratory in sterile isotonic medium containing gentamicin 20 ug/ml and maintained at 4° C. The maximum time from tumor procurement to initiation of vaccine protocol is 6 months.

Materials for Vaccine Preparation

Banking Medium. 450.0 ml RPMI without phenol red (Sigma catalogue # R-7509); 50.0 ml Human Serum Albumin (25% solution; final concentration=2.5%), and 5.0 ml glutamine (Sigma Chemical Co., catalog #G6392). Adjust to pH to 7.2 with 5. N NaOH. Sterile filter through 0.2 u filter into sterile plastic bottle attached to filtration unit (Nalgene—Fisher catalog # 09-740-25A).

Collagenase Solution (for making collagenase-coated lymphocytes for skin-testing). 100 ml Hanks+1% HA and 140 mg collagenase (Sigma catalogue # C-0130). Mix until completely dissolved. Sterile filter through 0.2 u filter.

Dinitrofluorobenzene (DNFB) Solution. (Reference: Miller and Claman, J Immunol 117:1519, 1976). Place 0.5 ml of 95% ethanol (USP grade—Pharmco Products) into a 50 ml beaker. Micropipet 65 μl of concentrated stock DNFB (Sigma D-1529) into the beaker. Mix by swirling for several minutes to get even suspension. Add 99.5 ml PBS (Mediatech Inc., catalogue # 21-031-CV) and a sterile stirring bar to a 250 ml beaker—then add DNFB suspension—rinse small beaker with PBS. Cover beaker with parafilm and stir overnight in 370 water bath. Filter through 0.2 u filter set into sterile plastic bottle. Cover bottle with aluminum foil, and store at 4° C.

Enzyme Solution For Tumor Dissociation. 100 ml Wash and Thaw Solution, 140 mg collagenase (Sigma catalogue # C-0130), and gentamicin stock solution—1. ml. Mix until completely dissolved. Sterile filter through 0.2 u filter.

Ethanol Solution For Fixation. 100% ethanol (USP grade—Pharmco Products)—100 ml. Water—100 ml. Sterile filter

Gentamicin Stock Solution (100×). 1 vial of gentamicin (40 mg/ml—2. ml=80 mg), 38. ml Hanks (Sigma catalog # 21-022-CV). Sterile filter through 0.2 u filter (final concentration of gentamicin=2 mg/ml).

Hanks+Gentamicin For Tumor Transport And Processing. Hanks (Sigma catalogue # 21-022-CV)—500 ml, and gentamicin stock solution—5. ml. Sterile filter through 0.2 u filter.

Hanks+Gentamicin For Skin Testing. 10 ml Hanks+Gentamicin for Tumor Transport and Processing. 10 ml Hanks—mix. Sterile filter through 0.2 u syringe filter

Hanks+0.1% HSA. 500 ml Hanks, 2.0 ml Human Serum Albumin (25% solution). Sterile filter through 0.2 u filter.

Hanks+1.0% HAS. 500 ml Hanks, 20. ml Human Serum Albumin (25% solution). Sterile filter through 0.2 u filter.

Hanks+EDTA (for lymphocyte separation). 500. ml Hanks (Sigma catalogue # 21-022-CV), add 0.5 g EDTA (Sigma catalogue # E-5134), and adjust pH to 7.2 with 5. N NaOH. Sterile filter through 0.2 u filter.

Sucrose Freezing Medium. Hanks balanced salt solution—60 ml, Human serum albumin (25% solution)—40 ml, Sucrose—8. g. Mix to dissolve completely. Sterile filter through 0.2 u filter. For skin testing, dispense 0.5 ml of Sucrose Freezing Medium per vial.

Sulfanilic Acid Diazonium Salt. Sulfanilic acid—anhydrous—Sigma—S-5643 (100 g), 10% Sodium nitrite—10 g sodium nitrite (Sigma S-3421), 100 ml water. Sterile filter through 0.2 u filter. Add 100 mg sulfanilic acid to 10. ml 0.1N hydrochloric acid (Sigma 210-4 (endotoxin-free)). Add ice-cold 10% sodium nitrite dropwise to sulfanilic acid—stir for 30 sec after each drop, then add droplet to starch-iodide paper until blue color appears (about 15 drops)—then stop (the final concentration of sulfanilic acid diazonium salt should be 40 mM). Sterile filter.

Wash & Thaw Solution. 500. ml Hanks, add 0.5 g EDTA, adjust pH to 7.2 with 5. N NaOH. Add 2.0 ml 25% Human Serum Albumin (final concentration=0.1%). Sterile filter through 0.2 u filter.

Tumor Processing

Briefly, cells are extracted by enzymatic dissociation with collagenase and by mechanical dissociation, frozen in a controlled rate freezer, and stored in liquid nitrogen until needed. Gentamicin 20 μg/ml is added to the tumor processing solution and washed out before the tumor cells are cryopreserved.

The tumor specimen is kept at 4° C. until processing—no more than 48 hours. Trim off and discard most of fat, connective tissue, and obviously necrotic material. Determine tumor weight. Add enough sterile Hanks+Gentamycin to cover bottom of a sterile Petri dish under the hood. Transfer the tumor tissue from the specimen container to the Petri dish. Cut off small sample of tumor (3-5 mm diameter) and place in vial with buffered formaldehyde; affix a prepared label. Mince tumor with scalpel so that pieces are 3-5 mm diameter. Pour minced tissue+liquid through sterile disposable filter set with sterilized steel screen: collect supernatant, pour into sterile tube (“TCM”). Keep at 4° C. until further processing.

Pipet appropriate amount of enzyme solution into disposable 125 ml or 250 ml flask with minced tumor pieces. Cap flask tightly and place in incubator shaker that has been pre-warmed to 37° C. Close the cover and set speed to about 350 RPM. Set timer on shaker for 30 minutes. After 30 minutes, turn off shaker and remove flask. Pipet fluid containing cell suspension into sterile mesh; transfer cell suspension to sterile 50 ml tube labeled “TCE”. Keep cell suspension at 4° C. until further processing. (The second digestion may be omitted if it appears that the only remaining tissue is connective tissue). Pipet enough enzyme solution to cover tissue pieces in disposable flask and place in incubator shaker for another 30 minutes at about 350 RPM. After 30 minutes, turn off shaker and remove flask. Pipet fluid containing cell suspension into sterile mesh; transfer cell suspension to sterile 50 ml tube labeled “TCE”. Pipet about 25 ml Hanks+Gentamycin into tumor dissociation flask; swirl briefly, then pipet as much of supernatant as possible through sterile mesh and add to “TCE” tube. Keep cell suspensions at 4° C. until further processing. Add Hanks (no gentamycin) to make volume of about 45 ml to each TCE tube.

Pellet the TCM and TCE tubes by centrifugation at 300 g (about 1100 RPM) for 7 minutes. Aspirate supernatants. Combine all resuspended TCE pellets in one 50 ml tube. Add about 45 ml Hanks (no gentamycin) and mix. Pellet the TCE tube by centrifugation at 300 g (about 1100 rpm) for 7 minutes. Aspirate supernatant and resuspend in Hanks (no gentamycin). Use at least 10 ml Hanks, but more can be added if pellet is very large. Resuspend the TCM pellet in 10 ml Hanks (no gentamycin).

Perform cell counts of TCE and TCM tubes according to Cell Counting Procedure. Following cell count, combine the TCE and TCM and label tubes as TC. Then, add enough Hanks (no gentamycin) to make volume of about 45 ml. Pellet cells by centrifugation at 300 g (about 1100 rpm) for 7 minutes. Aspirate supernatant.

Resuspend the cells in ice-cold Banking Medium, add the appropriate volume of 20% DMSO, and mix by inverting the capped tubes. Dispense the cell suspension into cryovials, and keep at 4° C. until ready to freeze. Freeze the cells in the programmed freezer and then place in liquid nitrogen bank. Cells should be maintained in the vapor phase of liquid nitrogen only.

Vaccine Preparation

Only if a sufficient number of mixed haptenized vaccine cells is obtained and the patient's vaccine passes lot release tests (endotoxin level <100 EU/ml, 14-day sterility testing negative), will patients be offered entry onto the study. Briefly, the vaccine consists of irradiated tumor cells, half of which have been haptenized with DNP and half with SA. The two types of haptenized cells are mixed in equal numbers, fixed with ethanol, and frozen. Melanoma cells may be admixed with variable numbers of tumor-associated lymphocytes and trace numbers of erythrocytes. The final volume of the vaccine is 0.2 ml.

A summary of the vaccine manufacturing procedure is as follows: The required number of autologous tumor cells will be thawed, washed, and divided into two aliquots. They will be irradiated to 2500 cGy. Then, one aliquot will be modified with dinitrophenyl (DNP) by the method of Miller and Claman (19) that we have used since 1988. This involves a 30-minute incubation of tumor cells with dinitrofluorobenzene under sterile conditions, followed by washing with Hanks solution. The second aliquot will be modified with sulfanilic acid (SA). The method is a modification of published procedures (Bach et al., J. Immunol., 121: 1460-1468, 1978; Sherman et al., J. Immunol., 121: 1432-1436, 1978; and Collotti et al., J. Exp. Med., 571-582, 1969). Cells are incubated for 5 minutes at room temperature with the diazonium salt of sulfanilic acid under sterile conditions, followed by washing with sterile Hanks solution. Following hapten modification, the cells are mixed 3:1 with 50% ethanol for a final concentration of 37.5%. Equal numbers of ethanol-treated DNP-modified and ethanol-treated SA-modified tumor cells will be mixed, washed, resuspended in cryopreservative (sucrose+human serum albumin) and dispensed in labeled vials. The vials are frozen by placing in a −86° freezer overnight, followed by transfer to and storage in liquid nitrogen. When a patient is ready to be treated, a vial of vaccine will be rapidly thawed, drawn up in a syringe, and injected intradermally within 20 minutes of thawing.

Specifically, thaw cryovials by placing in heating block at 37±0.5° until the contents are thawed with a few small ice crystals remaining. Gently pipet cell suspensions into 50 ml centrifuge tubes. Dilute DMSO in Wash & Thaw Solution, as follows: For each vial of cells in the tube, (a) add 0.05 ml & swirl for 30 sec, then (b) add 0.1 ml & swirl for 30 sec, then (c) add 0.2 ml & swirl for 30 sec, then (d) add 0.4 ml & swirl for 30 sec, then (e) add 0.8 ml & swirl for 30 sec. Allow cells to sit at room temperature for 5 minutes. To each 50 ml centrifuge tube add ml Wash & Thaw solution: 10 ml for each original vial of cells in the tube. Pellet cells by centrifugation at 300 g (about 1100 RPM) for 7 minutes. Aspirate supernatants. Suspend one pellet in 10 ml Hanks+0.1% HSA. Then resuspend and consolidate all of the pellets into one 15 ml centrifuge tube with an affixed patient label. Do cell count as per Cell Counting Procedure.

Irradiation. Pellet cells by centrifugation at 300 g (about 1100 RPM) for 7 minutes. Aspirate supernatant and suspend pellet in 2 4 ml (depending on pellet size) of Hanks+0.1% HSA. Pipet the cell suspension to cryovials, about 2. ml per cryovial, and place in refrigerated block. Irradiate tumor cells in cesium irradiator to 2500 cGy (at the currently calculated dose rate of 106.3 cGy/min, the time is 23.5 minutes).

After irradiation, pipet cells into 15 ml centrifuge tubes. Add 10 ml Hanks-no HSA and mix. Pellet cells by centrifugation at 300 g (about 1100 RPM) for 7 minutes. Aspirate supernatant and resuspend pellet in 10 ml Hanks-no HSA. Perform cell count as per Cell Counting SOP, except: Do not add trypan blue. Count large and small nucleated cells.

Unmodified Skin Test Materials. Pipet 15×10⁶ tumor cells into tube labeled “ST-UN”. Pellet the “ST-UN” tube by centrifugation at 300 g (about 1100 RPM) for 7 minutes. Aspirate the supernatant and resuspend pellet in 1. ml cold (4° C.) Hanks with 1% HSA. Add 3. ml of ice-cold 50% ethanol to the “ST-UN” tube while vortexing at low speed. Incubate the tube at 4° for 10 minutes. Pellet cells by centrifugation at 300 g (about 1100 RPM) for 7 minutes. Aspirate supernatant and resuspend in 2. ml Hanks+1% HSA. Perform cell count of “ST-UN” tube by Cell Counting Procedure, except do not add trypan blue. Count only large and small cells.

Pellet “ST-UN” tube by centrifugation at 300 g (about 1100 RPM) for 7 minutes. Aspirate supernatant. Resuspend “ST-UN” pellet in volume of ml Sucrose Freezing Medium to make a concentration of 1×10⁶ large cells per 0.15 ml (6.7×10⁶/ml). Dispense “ST-UN” cells into vials, 0.15 ml/vial (3×10⁶ tumor cells/vial) Place cryovials at 4° C. until ready for freezing.

Hapten Modification. Divide remainder of tumor cell suspension into two equal aliquots. Label one tube “DNP” and the other “SA”. Pellet both cell suspensions by centrifugation at 300 g (about 1100 RPM) for 7 minutes. Aspirate supernatants. To the SA tube, add 2. ml Hanks-no HSA and keep at 4° C. until needed.

To the “DNP” tube add Hanks without albumin to bring the concentration of cells (tumor cells+lymphocytes) to 5×10⁶/ml. For each 1.0 ml of cell suspension, add 0.1 ml of DNFB solution. Mix and incubate at room temperature for 30 minutes; gently mix every 10 minutes.

While the DNP cells are incubating, dilute the diazonium salt of SA 1:8 in Hanks without albumin. Adjust the pH to 7.2 by dropwise addition of IN NaOH (2-3 drops). Sterile filter the solution. Pellet the “SA” tube by centrifuging at 300 g (about 1100 RPM) for 7 minutes. Aspirate supernatant. Resuspend the pellet in a quantity of the diluted diazonium salt to make a cell concentration (intact TC+LY+dead) of 5×10⁶/ml. Immediately resuspend. Incubate for 5 minutes at room temperature.

As soon as the DNP and SA are finished (30 minutes and 5 minutes, respectively), stop the reactions by adding 0.5 ml of the stock solution of human serum albumin (25% solution) to the tube, capping, and mixing. Pellet cells by centrifugation at 300 g (about 1100 RPM) for 7 minutes. Wash the cells twice in Hanks+1.0% HSA.

Ethanol Treatment. After the last centrifugation, resuspend the cells in the DNP and SA tubes in 1. ml ice-cold (4o) Hanks with 1% HSA. Add 3. ml of ice-cold 50% ethanol to each tube while vortexing at low speed. Incubate the tubes at 4° C. for 10 minutes. Pellet cells by centrifugation at 300 g (about 1100 RPM) for 7 minutes. Aspirate supernatant, resuspend in 10 ml Hanks+1% HSA, and pellet by centrifugation at 300 g (about 1100 RPM) for 7 minutes. Aspirate supernatant and resuspend in 2. ml Hanks+1% HSA. Perform cell count of SA and DNP tubes (do not add trypan blue). Count large and small nucleated cells and erythrocytes.

To determine the proportion of dead cells: Add one drop of suspension from SA and DNP tubes to separate glass slides. Add one drop trypan blue to each slide. Place a cover slip over the drops. Perform a count of trypan-blue (+) and trypan blue (−) cells by counting 100 cells. Calculate the percentage of trypan-blue (+) cells and record in the batch record.

Haptenized Skin Test Materials. Remove 4×10⁶ large cells from SA tube and pipet into tube with affixed patient label and label “ST-SA”. Remove 4×10⁶ large cells from DNP tube and pipet into tube labeled “ST-DNP”. Pellet cells in both tubes by centrifugation at 300 g (about 1100 RPM) for 7 minutes. Aspirate supernatants. Resuspend each in 0.60 ml Sucrose Freezing Medium. Add 0.15 ml of ST-SA cells to each of 4 cryovials. Add 0.15 ml of ST-DNP cells to each of 4 cryovials. Place cryovials at 4° C. until ready for freezing.

Combining and Aliquotting SA and DNP-Haptenized Vaccine. Calculate number of remaining SA-modified and DNP-modified tumor cells. Mix equal numbers (maximum possible) of remaining SA-modified and DNP-modified cells in a tube with an affixed patient label. Pellet cells by centrifugation at 300 g (about 1100 RPM) for 7 minutes. Aspirate supernatant. Calculate the total number of SA-modified+DNP-modified tumor cells. Resuspend the pellet in Sucrose Freezing Medium.

Gently mix the vaccine cell suspension. Add 0.2 ml of the vaccine suspension to each of the pre-labeled “VACC”. Freeze all of the vials by placing them into Nalgene Cryo 1° C. Freezing Container with isopropanol in −86° C. freezer. Leave vials overnight. Then transfer to liquid nitrogen bank.

Pre-Vaccine Skin-Testing

This is performed 2 weeks prior to beginning vaccine injections by the intradermal injection of 0.15 ml of test material on the forearm. DTH is assessed at 48 h by measuring the mean diameter of induration. Patients are tested for DTH to the following materials:

1) 1.0×10⁶ autologous melanoma cells: irradiated (2500 cGy), DNP-modified, fixed

2) 1.0×10⁶ autologous melanoma cells: irradiated (2500 cGy), SA-modified, fixed

3) 1.0×10⁶ autologous melanoma cells: irradiated (2500 cGy), unmodified, fixed

4) diluent—Hanks solution with sucrose+human serum albumin (HSA)

All skin test materials will be prepared and frozen in advance of the date of testing. The standard operating procedure is appended. An aliquot of each material will be tested for sterility and endotoxin and the material will be used only if it passes both tests (no growth in 14-day sterility assay and endotoxin level <100 EU/ml).

Patients who have a negative baseline DTH reaction (<5 mm induration) to all three of the melanoma cell preparations will continue on the study to receive vaccine at one of the three study doses. Patients who have a positive baseline DTH reaction (≧5 mm induration) to any of the three melanoma cell preparations will be eligible to receive vaccine only at dosage level B (0.5×10⁶ tumor cells).

Method for Skin Test Application and Measurement. After the patient has arrived, thaw a vial of each of the cellular materials. The thawed materials may be stored at 4° C. for 20 minutes prior to injection. The most proximal skin test should be at least 3 cm below the elbow crease on the ventral forearm and each injection should be separated by at least 3 cm. If one of the patient's forearms is unusable, e.g., because of post-surgical lymphedema, all skin test must be done on the same arm by using medial and lateral edges of the ventral forearm.

For each cellular skin test material, draw up the contents (0.15 ml) into a 0.5 cc Lo-Dose insulin syringe and inject intradermally, making sure that a wheal is raised by the injection. For soluble skin test materials (PPD, diluent, gentamicin) draw up 0.10 ml.

Measuring the Reactions. After 48±4 hours, inspect the skin test injection sites. Measure the diameters of erythema at each site, i.e., the longest diameter and the diameter perpendicular to this. Palpate each reaction to determine the induration. Measure the diameters of induration at each site; the longest diameter and the one perpendicular to this. A positive response is defined by mean diameter of induration ≧5 mm.

Vaccine Administration

The left arm is the site of all vaccine injections, unless the patient has had a left axillary lymph node dissection; in that case the right arm will be used for all vaccine injections. If a patient has undergone bilateral axillary dissections, the vaccine injections are made on the left upper thigh. See diagram.

| Vaccine #1 | ventral forearm | | Vaccine only | | |
|---|---|---|---|---|---|
| Vaccine #2 | dorsal upperarm | BCG-A only | BCG-A + | BCG-A + | BCG-A + |
| | | | vaccine | vaccine | vaccine |
| Vaccine #3 | dorsal upperarm | | BCG-A + | BCG-A + | BCG-A + |
| | | | vaccine | vaccine | vaccine |
| Vaccine #4 | dorsal upperarm | | BCG-B + | BCG-B + | BCG-B + |
| | | | vaccine | vaccine | vaccine |
| Vaccine #5 | dorsal upperarm | | BCG-B + | BCG-B + | BCG-B + |
| | | | vaccine | vaccine | vaccine |
| Vaccine #6 | dorsal upperarm | | BCG-C + | BCG-C + | vaccine only |
| | | | vaccine | vaccine | |
| Vaccine #7 | dorsal upperarm | | BCG-C + | BCG-C + | BCG-C + |
| | | | vaccine | vaccine | vaccine |
| Vaccine #8 | dorsal upperarm | | BCG-C + | BCG-C + | BCG-C + |
| | | | vaccine | vaccine | vaccine |

On day 1, patients are injected intradermally on the ventral forearm with mixed haptenized vaccine without added BCG. This serves as an induction dose of vaccine. Draw up the vaccine suspension (0.2 ml, tumor cells in Hanks solution with sucrose and human serum albumin) into a 0.5 cc “Lo-Dose” insulin syringe and inject intradermally into the mid ventral forearm.

On day 8+1, patients will receive cyclophosphamide 300 Mg/M² as a bolus injection over 5 minutes. The rationale is based on published evidence from animal and clinical studies (Hengst et al., Cancer Res, 40: 2135-2141, 1980; Berd et al., Cancer Res, 46:2572-2577, 1986) showing that cyclophosphamide augments the development of cell-mediated immunity to tumor-associated antigens. Cyclophosphamide is reconstituted with bacteriostatic water for injection, USP, at a dilution of 20 mg of cyclophosphamide per 1 ml of water.

Three days later the patients is injected intradermally on the dorsal upper arm with vaccine mixed with BCG and this will be repeated weekly for a total of 6 weeks. The injection of vaccines #2-7 will be made into the same limb as the induction dose. Three dose ranges of mixed haptenized vaccine will be studied. The method of administration of vaccine #2-7 is as follows: Prepare BCG by reconstituting with 1.0 ml saline for injection (without preservative) according to package label. Prepare 1:10, 1:100, and 1:1,000 dilutions of the BCG in saline for injection and label as A, B, and C, respectively. After the patient has arrived, thaw a vial of mixed haptenized vaccine, checking for identifying information. Add 0.1 ml of the proper dilution of BCG (see below) to the vaccine suspension. Immediately draw up the vaccine-BCG mixture into a 0.5 cc “Lo-Dose” insulin syringe and inject intradermally into three adjacent sites, separated by about 1 cm, on the upper arm.

The administration of vaccines #2 and #6 is modified to allow a better assessment of toxicity. Vaccine #2: The administration of the vaccine is given as described. However, an additional dose of BCG will be administered to differentiate the local toxicity of BCG from the local toxicity of the vaccine-BCG combination. This is done as follows: Add 0.1 ml of BCG dilution “A” (1:10) to a sterile vial. Add 0.2 ml of saline for injection to the vial. Mix and withdraw 0.1 ml with a 0.5 cc “Lo-Dose” insulin syringe. Inject intradermally about 1 cm medial to the most medial vaccine injection site. Vaccine #6: Two-thirds of the vaccine dose will be injected with BCG and one-third without BCG. This is done as follows: Gently mix the thawed vaccine suspension and draw up 0.07 ml into a 0.5 cc “Lo-Dose” insulin syringe. Inject the vaccine intradermally into the most lateral of the three intended vaccine sites. Add 0.1 ml of the proper BCG dilution (“C”, 1:1,000) to the remainder of the vaccine suspension and inject intradermally into two sites medial to the first injection.

Booster Injections

Patients who have not exhibited tumor progression and who have not received other melanoma treatments in the interval will be given a booster vaccine at the six month point (measured from beginning the vaccine program) if sufficient cells are available. The dose and method of administration of the booster injections will be the same as vaccine #7.

Assignment of Vaccine Dose

Patients whose baseline DTH response to autologous melanoma cells was negative (<5 mm induration) are assigned to one of three vaccine dosage levels.

A=5.0×10⁴ tumor cells

B=5.0×10⁵ tumor cells

C=5.0×10⁶ tumor cells

A patient is assigned to one of these dosage levels according to the yield of mixed haptenized, fixed tumor cells obtained after vaccine production. If the yield of tumor cells is ≧2×10⁶ and <20×10⁶, the dose assignment is “A”. If the yield of tumor cells is ≧20×10⁶ and <55×10⁶, the dose assignment is “B”. If the yield of tumor cells is ≧55×10⁶, the dose assignment is “C”. The three dosage levels will be tested simultaneously. At least 6 and no more than 14 evaluable patients will be treated at each dosage level. After 14 evaluable patients have been treated at a given dosage level, subsequent patients are assigned to the next unfilled dosage level.

Patients who have a positive (≧5 mm induration) baseline DTH response to any of the melanoma cell preparations will be eligible to receive the vaccine at dosage level B only. If their vaccines had been aliquotted and frozen for dosage levels A or C, they will not receive vaccine treatment and will be discontinued from the study. A maximum of 14 patients with positive baseline DTH reactions will be treated.

BCG Doses

The first dose (induction dose) contains no BCG. The second and third vaccines are mixed with 0.1 ml of a 1:10 dilution of Tice BCG (“Tice-A”). The fourth and fifth vaccines are mixed with 0.1 ml of a 1:100 dilution (“Tice-B”). The sixth and seventh and the booster vaccines are mixed with 0.1 ml of a 1:1000 dilution (“Tice-C”). The ideal vaccine reaction is an inflammatory papule with no more than small (<5 mm) central ulceration. If reactions are larger than this, the dose of BCG is further attenuated ten-fold.

Post-Vaccine Skin-Testing

This is performed by the intradermal injection of test material on the forearm, and DTH is assessed at 48 h by measuring the mean diameter of induration. Patients are tested for DTH to the following materials:

1) 1.0×10⁶ autologous melanoma cells: irradiated (2500 cGy), DNP-modified, fixed

2) 1.0×10⁶ autologous melanoma cells: irradiated (2500 cGy), SA-modified, fixed

3) 1.0×10⁶ autologous melanoma cells: irradiated (2500 cGy), unmodified, fixed

4) 5.0×10⁶ autologous peripheral blood lymphocytes, unmodified, fixed

5) 5.0×10⁶ autologous peripheral blood lymphocytes—coated with collagenase, fixed

6) diluent—Hanks solution with sucrose+human serum albumin (HSA)

7) gentamicin 1.0 μg in 0.1 ml Hanks solution

8) PPD intermediate

All skin test materials (with the exception of PPD, which is commercially available and approved for human testing) are prepared and frozen in advance of the date of testing. The volume of the cellular materials (#1-5) is 0.15 ml; the volume of materials #6-8 is 0.10 ml. The procedure for measuring and photographing DTH reactions is as described above.

Tumor Inflammation

Patients are evaluated clinically to determine whether they developed inflammation in superficial metastases (dermal and subcutaneous). This is defined as erythema in and/or around tumor sites that develops following vaccine treatment. Metastases that exhibit inflammation are photographed and are biopsied if possible.

Clinical Evaluation of Patients

Anti-tumor responses are documented. Only patients with measurable metastases at the time of beginning vaccine treatment are assessed for response. CT or MRI imaging are performed every 3 months until tumor progression. Standard definitions of response will be used:

Complete Response (CR): Complete disappearance of all clinically detectable disease by two observations no less than 4 weeks apart.

Partial Response (PR): A ≧50% decrease (in bidimensional lesions) or ≧30% decrease (in unidimensional lesions) in the total tumor size of the lesions (as determined by the sum of the products of the two greatest perpendicular diameters of all measurable lesions), which have been measured to determine the effect of therapy. The decrease is documented by two observations no less than 4 weeks apart. In addition, there are no appearance of new lesions or progression of any lesion.

Stable Disease (SD): A <50% decrease in bidimensional lesions or <30% decrease in unidimensional lesions (as defined above) or, a <25% increase in any individual lesions for a least 4 weeks.

Progressive Disease (PD): An increase of ≧25% of one or more measurable lesions or the appearance of any new lesion.

### Example 9

### Correlation Between Vaccine Dose and DTH Response

This Example relates to the correlation between DTH response, which is an established indicator of clinical response in immunotherapy, and the amount of tumor cells in the vaccine. The Example shows that preparing immunotherapy vaccines based on the total number of tumor cells rather than live tumor cells enables a much better prediction of immune response, and thereby clinical outcome.

Tumor cell vaccines based on DNP-modified autologous melanoma cells were prepared as described in published PCT application Nos. WO 96/40173, WO 00/29554, WO 00/09140, WO 00/38710, WO 00/31542, WO 99/56773, WO 99/52546, WO 98/14206, and in U.S. Pat. Nos. 5,290,551; 6,248,585; and 6,333,028. Using Trypan Blue exclusion, both the number of live, i.e., Trypan Blue excluding, and dead, i.e., non-Trypan Blue-excluding but with a substantially intact shape, cells in each vaccine does were counted.

In the majority of the patients, the treatment schedule was as follows: On Day 0, an induction dose of about 0.5×10⁶-1×10⁶ live, DNP-modified cells was administered, followed at Day 7 by an intravenous injection of about 300 mg/M² cyclophosphamide. On Day 10, a tumor cell vaccine comprising about 2.0×10⁶-25.0×10⁶ live, DNP-modified tumor cells was injected intradermally. In most patients, another 5 doses of DNP-modified tumor cells were administered at weekly intervals.

Then, the DTH-response of each patient to unmodified autologous tumor cells was measured as described in the aforementioned WO publications and U.S. Patents, and in Example 6. Briefly, about 1×10⁶ Trypan-Blue excluding tumor cells were injected intradermally on the patient's forearm. Control material (diluent=Hanks+HSA) was similarly injected. After 48 hours the patient's arm was inspected. For each injection site, the largest diameter of induration was measured (in millimeter) with a ruler.

The resulting data was plotted as DTH response (in mm) versus the number of live tumor cells or DTH response versus the total number of tumor cells (i.e., both live and dead cells), in each vaccine dose, and subjected to linear regression analysis. The total number of tumor cells, i.e., live plus dead, yielded a better correlation to DTH response (R=0.222; p<0.001), and thereby clinical response, than live cells only (R=0.033; p=0.608).

### Example 10

### Contribution of Dead Cells to Vaccine Effectiveness

Treatment of melanoma patients with a vaccine consisting of autologous tumor cells modified with the hapten dinitrophenyl (DNP) and preceded by low dose cyclophosphamide induces delayed-type hypersensitivity (DTH) to autologous, unmodified tumor cells. This DTH response is a significant predictor of survival.

The present Example describes the analysis of vaccines prepared for 284 patients who were treated following resection of regional or distant metastases to determine whether the dose and composition correlated with immunological response. Briefly, regression analysis showed no significant association between the magnitude of DTH and the number of intact (trypan blue-excluding) melanoma cells per dose, while vaccines containing higher numbers of dead tumor cells or higher proportions of dead tumor cells induced better immune responses. Thus, dead tumor cells contribute to the immunogenicity of the DNP-vaccine.

Production of Autologous, DNP-Modified Vaccine

Metastatic tumor was excised, maintained at 4° C., and delivered to the laboratory within 48 hours of excision. Tumor cells were extracted by enzymatic dissociation with collagenase and DNAse, aliquotted, frozen in a controlled rate freezer, and stored in liquid nitrogen in a medium containing human albumin and 10% dimethylsulfoxide until needed. On the day that a patient was to be treated, an aliquot of cells was thawed, washed, and irradiated to 2500 cGy. Then they were washed again and modified with DNP by the method of Miller and Claman (Miller J. Immunol. 1976; 117:1519-1526). After washing, the cells were counted, suspended in 0.2 ml Hanks solution with human albumin, and maintained at 4° C. until administered.

Vaccine Administration

Just prior to injection, 0.1 ml of Tice BCG was added to the vaccine. Then the mixture as drawn up in a 1. ml syringe and injected intradermally, usually into the upper arm, excluding the arm ipsilateral to a lymph node dissection.

Five vaccine dosage-schedules were tested sequentially. All dosage-schedules included the administration of low dose (300 mg/M2) cyclophosphamide, a cytotoxic drug that augments cell-mediated immunity when administered at the proper time in relation to immunization. Moreover, in all dosage-schedules, the dose of BCG was progressively attenuated to produce a local reaction consisting of an inflammatory papule without ulceration.

Delayed-Type Hypersensitivity (DTH) Responses Induced by DNP-Vaccine

DTH testing was performed pre-treatment and at various times post-treatment. Test materials consisted of autologous cryopreserved melanoma cells, either DNP-modified or unmodified; autologous peripheral blood lymphocytes (PBL); and PPD. A positive response was defined as a mean diameter of induration ≧5 mm, measured after 48 hours.

DTH studies have been performed in two types of patients: 1) Stage IV melanoma with surgically-incurable metastases (N=83), and 2) Stage III or IV post-surgical adjuvant melanoma patients, i.e., clinically melanoma free following resection of one or more metastases (N=284). Almost all (99%) patients developed a large (median diameter=24 mm) PPD response, which indicates that they were sufficiently immunocompetent to respond to a strong antigen. Most patients (95%), with measurable metastases or tumor-free, also exhibited a large (median diameter=17 mm) DTH response to DNP-modified autologous melanoma cells. A much lower proportion of patients (57%) developed DTH to unmodified autologous melanoma cells, and the median diameter was 5 mm. However, this parameter is the most clinically meaningful because it is predictive of survival. For example, in the post-surgical adjuvant group, the development of a positive response to unmodified tumor cells was associated with significantly greater 5-year survival (71% vs. 49%) (p<0.001, log rank test).

Effect of Vaccine Dose and Composition on Induction of DTH Responses

Table 5 shows a summary of the composition of all of the vaccines administered. All vaccines contained intact (trypan blue-excluding) tumor cells, dead (trypan blue positive) tumor cells, and lymphocytes. As seen in the table, there was considerable variation in vaccine composition among patients. However, for a given patient the composition of multiple vaccines manufactured over a period time was similar. Therefore, for all analyses, the mean value for each patient was used.

TABLE 5
Composition of Vaccines

| Dose Parameter | median (range) |
|---|---|
| No. Live Tumor Cells (× 10⁶) | 6.8 (0.5-25.0) |
| No. Dead Tumor Cells (× 10⁶) | 8.0 (0.1-71.2) |
| No. Live + Dead Tumor Cells (× 10⁶) | 16.6 (0.5-73.0) |
| % Live Tumor Cells | 44% (3%-88%) |
| % Lymphocytes | 34% (0-86%) |

Using linear regression analysis, it was determined whether the maximum DTH response to autologous unmodified melanoma cells was dependent on the dose of intact tumor cells. No significant relationship was observed (adjusted squared multiple R=0.000, p=0.512). Next, the effect of increasing numbers of dead tumor cells on the development of DTH was analyzed. Surprisingly, there was a small but significant positive relationship between the mean number of dead cells in the vaccines of a given patient and that patient's maximum DTH to unmodified melanoma (adjusted squared multiple R=0.060, p<001). There was a significant inverse relationship between DTH and the proportion of intact tumor cells per dose (calculated as the number of intact tumor cells divided by the total number of tumor cells) (adjusted multiple squared R=0.063, p<0.001).

These analyses were confirmed by the observation that patients whose vaccines contained >50% live cells developed significantly smaller DTH responses than patients whose vaccines contained 26-50% or ≦25% live cells. Thus, only 37% of patients whose vaccines contained >50% live cells developed DTH to unmodified melanoma, as compared with 69% and 65% of patients whose vaccines contained ≦25% or 26-50% live cells, respectively (p<0.001, Kruskal-Wallis test).

Survival using the Kaplan-Meier method in which patients were stratified by each of the vaccine composition parameters was also conducted. None of these parameters had any significant effect on relapse-free or overall survival.

### Discussion

Our previous studies have demonstrated that the efficacy of autologous, DNP-modified melanoma vaccine is dependent on the induction of DTH to autologous, unmodified melanoma cells. However, the intensity of the DTH response to autologous, unmodified melanoma cells was not primarily determined by the dose of vaccine administered, at least over the dosage range (0.5-25.0×106) that we tested. We have defined the dose by the number of melanoma cells that were live, i.e. excluding the supravital dye, trypan blue, although rendered proliferation incompetent by irradiation and DNP modification.

There was a direct correlation between DTH and the number of dead cells per dose. The number of dead cells per dose accounted for about 6% of the variation in DTH responses. That it is biologically significant is reinforced by the observation that DTH responses were greater in patients whose vaccine had the lowest proportions of live cells. Therefore, the data shows that dead tumor cells contribute to the immunogenicity of the DNP-modified vaccine, and the results are applicable to other cellular human cancer vaccines.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are to some degree approximate, and are provided for purposes of description.

Patents, patent applications, and publications are cited throughout this application, the disclosures of which are incorporated herein by reference in their entireties.

What is claimed is:

1. A method of preserving tumor cells, which method comprises:
contacting the tumor cells with ethanol at a concentration effective to preserve the tumor cells;
whereby the tumor cells are better preserved than the same type of tumor cells incubated in control medium without ethanol for the same period of time and at the same temperature.

2. The method of claim 1, wherein the concentration of ethanol is within the range of about 22.5% to about 75% by volume.

3. The method of claim 2 wherein the concentration of ethanol is about 37.5% by volume.

4. The method of claim 1 wherein the tumor cells are contacted with ethanol for a period of about 2 minutes to about 24 hours at a temperature within the range of about 0° C. to about 20° C.

5. The method of claim 4 wherein the tumor cells are contacted with ethanol for a period of about 10 minutes at a temperature of about 4° C.

6. The method of claim 1 wherein the tumor cell preservation comprises preservation of antigenicity.

7. The method of claim 1, wherein the tumor cell preservation comprises preservation of the number of cells.

8. The method of claim 1, wherein the tumor cells are selected from the group consisting of melanoma cells, ovarian cancer cells, colorectal cancer cells, small cell lung cancer cells, kidney cancer cells, breast cancer cells, and leukemia cells.

9. The method of claim 8, wherein the tumor cells are melanoma cells.

10. The method of claim 8, wherein the tumor cells are ovarian cancer cells.

11. The method of claim 1, wherein the tumor cells are conjugated to a hapten.

12. The method of claim 11, wherein the hapten is selected from the group consisting of DNP, TNP, and sulfanilic acid.

13. A composition comprising tumor cells for use in a vaccine and a concentration of ethanol effective to preserve the tumor cells.

14. The composition of claim 13, wherein the concentration of ethanol is within the range of about 22.5% to about 75% by volume.

15. The composition of claim 14 wherein the concentration of ethanol is about 37.5% by volume.

16. The composition of claim 13 wherein the temperature of the composition is within the range of about 0° C. to about 20° C.

17. The composition of claim 16 wherein the temperature is about 4° C.

18. The composition of claim 13, wherein the concentration of ethanol is effective to preserve the antigenicity of the tumor cells.

19. The composition of claim 13, wherein the concentration of ethanol is effective to preserve the number of tumor cells.

20. The composition of claim 13, wherein the tumor cells are selected from the group consisting of melanoma cells, ovarian cancer cells, colorectal cancer cells, small cell lung cancer cells, kidney cancer cells, breast cancer cells, and leukemia cells.

21. The composition of claim 20, wherein the tumor cells are melanoma cells.

22. The composition of claim 20, wherein the tumor cells are ovarian cancer cells.

23. The composition of claim 13, wherein the tumor cells are conjugated to a hapten.

24. The composition of claim 21, wherein the hapten is selected from the group consisting of DNP, TNP, and sulfanilic acid.

25. A tumor cell vaccine comprising
(i) dead autologous tumor cells; and
(ii) an adjuvant,
wherein the vaccine is essentially free of live autologous tumor cells of the same tumor type.

26. The tumor cell vaccine of claim 25, wherein the antigenicity of the dead autologous tumor cells is no less than the antigenicity of live autologous tumor cells of the tumor same type.

27. The tumor cell vaccine of claim 25, wherein the tumor cells are selected from the group consisting of melanoma cells, ovarian cancer cells, colorectal cancer cells, small cell lung cancer cells, kidney cancer cells, breast cancer cells, and leukemia cells.

28. The tumor cell vaccine of claim 25, wherein the tumor cells are melanoma cells.

29. The tumor cell vaccine of claim 25, wherein the tumor cells are ovarian cancer cells.

30. The tumor cell vaccine of claim 25, wherein the tumor cells are conjugated to a hapten.

31. The tumor cell vaccine of claim 30, wherein the hapten is selected from the group consisting of DNP, TNP, and sulfanilic acid.

32. A method for treating cancer in a subject, the method comprising administering a vaccine comprising an adjuvant and autologous tumor cells which have been treated to render them dead, wherein the vaccine is essentially free of live autologous tumor cells of the same tumor type.

33. The method of claim 32, wherein the tumor cells have been treated with ethanol.

34. The method of claim 33, wherein the tumor cells have been treated with an ethanol concentration within the range of about 22.5% to about 75% by volume.

35. The method of claim 34 wherein the tumor cells have been treated with an ethanol concentration of about 37.5% by volume.

36. The method of claim 32, wherein the tumor cells are conjugated to at least one hapten.

37. The method of claim 36, wherein the at least one hapten is selected from the group consisting of DNP, TNP, and sulfanilic acid.

38. The method of claim 37, wherein the tumor cells comprises a first fraction of tumor cells conjugated to DNP, and a second fraction of tumor cells conjugated to sulfanilic acid.
